(19) United States
(12) Patent Application Publication
(10) Pub. No.: US 2015/0216975 A1
(43) Pub. Date: Aug. 6, 2015

(54) NANOVECTOR BASED DRUG DELIVERY SYSTEM FOR OVERCOMING DRUG RESISTANCE

(71) Applicant: WILLIAM MARSH RICE UNIVERSITY, Houston, TX (US)

(72) Inventors: David S. Baskin, Houston, TX (US); Daniela Marcano, Houston, TX (US); Martyn A. Sharpe, Houston, TX (US); James M. Tour, Bellaire, TX (US)

(73) Assignees: William Marsh Rice University, Houston, TX (US); The Methodist Hospital Research Institute, Houston, TX (US)

(21) Appl. No.: 14/436,127

(22) PCT Filed: Mar. 15, 2013
(86) PCT No.: PCT/US13/32502

§ 371 Date: Apr. 16, 2015

Related U.S. Application Data
(60) Provisional application No. 61/714,478, filed on Oct. 16, 2012.

(51) Int. Cl. A61K 47/10; A61K 47/48; A61K 31/405; A61K 31/517; G01N 33/574; A61K 31/451

(52) U.S. Cl. CPC A61K 47/10; G01N 33/57492; A61K 31/451; A61K 31/405; A61K 31/517; A61K 47/48384; A61K 47/48246; G01N 2333/71; G01N 2333/7155

(57) ABSTRACT

Various embodiments of the present invention provide therapeutic compositions for specifically targeting tumor cells. In some embodiments, the therapeutic compositions generally include: (1) a plurality of nanovectors; (2) one or more active agents associated with the nanovectors, where the one or more active agents have activity against the tumor cells; (3) one or more active agent enhancers associated with the nanovectors; and (4) one or more targeting agents associated with the nanovectors, where the one or more targeting agents have recognition activity for one or more markers of the tumor cells. Additional embodiments of the present invention pertain to methods of targeting tumor cells in a subject by administering one or more of the aforementioned therapeutic compositions to the subject. Further embodiments of the present invention pertain to methods of formulating the aforementioned therapeutic compositions for targeting tumor cells in a subject in a personalized manner.

### CROSS-REFERENCE TO RELATED APPLICATIONS

This application claims priority to U.S. Provisional Patent Application No. 61/714,478, filed on Oct. 16, 2012. The entirety of the aforementioned application is incorporated herein by reference.

This application is related to Patent Cooperation Treaty Application No. PCT/US2012/35267, filed on Apr. 26, 2012, which claims priority to U.S. Provisional Patent Application No. 61/479,220, filed on Apr. 26, 2011. This application is also a related to Patent Cooperation Treaty Application No. PCT/US2010/54321, filed on Oct. 27, 2010, which claims priority to U.S. Provisional Application No. 61/255,309, filed on Oct. 27, 2009. This application is also a related to Patent Cooperation Treaty Application No. PCT/US2008/078776, filed on Oct. 3, 2008, which claims priority to U.S. Provisional Application No. 60/977,311, filed on Oct. 3, 2007. The entirety of each of the aforementioned applications is incorporated herein by reference.

### STATEMENT REGARDING FEDERALLY SPONSORED RESEARCH

Not applicable.

### BACKGROUND

Current methods to treat various types of tumors suffer from various limitations. Such limitations include an inability to effectively and specifically deliver desired drugs to tumor sites. Such limitations are further escalated when desired drugs are hydrophobic, and when the tumor displays resistance to multiple drugs. Additional obstacles include lack of effective methods of making personalized drug delivery compositions that effectively target a desired tumor in a particular subject. Therefore, more efficient and effective approaches to targeted drug delivery are desired for treating various types of tumors.

### SUMMARY

In some embodiments, the present disclosure provides therapeutic compositions for targeting tumor cells. In some embodiments, the therapeutic compositions generally include: (1) a plurality of nanovectors; (2) one or more active agents associated with the nanovectors, where the one or more active agents have activity against the tumor cells; (3) one or more active agent enhancers associated with the nanovectors; and (4) one or more targeting agents associated with the nanovectors, where the one or more targeting agents have recognition activity for one or more markers of the tumor cells. In some embodiments, the one or more active agents and the one or more active agent enhancers are associated with the same nanovector molecules. In some embodiments, the one or more active agents and the one or more active agent enhancers are associated with different nanovector molecules.

In some embodiments, the nanovectors may include an ultra-short single-walled carbon nanotube that is functionalized with a plurality of solubilizing groups. In some embodiments, the nanovectors may include polyethylene glycol functionalized hydrophilic carbon clusters (PEG-HCC).

In some embodiments, the one or more active agents may include one or more anti-cancer agents, such as vinblastine, docetaxel, and combinations thereof. In some embodiments, the one or more active agent enhancers may include one or more drug transport pump inhibitors, such as xenobiotic drug pump inhibitors. In some embodiments, the one or more markers may include a receptor on a surface of tumor cells, such as epidermal growth factor receptors, interleukin receptors, and combinations thereof.

In some embodiments, the one or more targeting agents may include at least one of antibodies, proteins, peptides, RNA, DNA, aptamers, small molecules, dendrimers, and combinations thereof. In some embodiments, the one or more targeting agents may include an antibody, a peptide or a small molecule.

Additional embodiments of the present disclosure pertain to methods of targeting tumor cells in a subject by administering one or more of the aforementioned therapeutic compositions to the subject. In some embodiments, the subject is a human being suffering from cancer. In some embodiments, the administering of the therapeutic composition may occur by intravenous administration.

Further embodiments of the present disclosure pertain to methods of formulating one or more of the aforementioned therapeutic compositions for targeting tumor cells in a subject in a personalized manner. In some embodiments, such methods include: (1) determining expression levels of one or more markers of the tumor cells; and (2) formulating a therapeutic composition based on the determined expression levels of the one or more markers. In some embodiments, such methods may also include a step of determining the susceptibility of the tumor cells to various active agents, and selecting one or more active agents based on the determined susceptibility of the tumor cells to those active agents. In some embodiments, the susceptibility of the tumor cells to the active agents may be determined in the presence of one or more active agent enhancers.

As set forth in more detail herein, the methods and compositions of the present disclosure can be used to effectively and specifically target various types of tumors. In some embodiments, the targeted tumor cells may be associated with at least one of cervical cancer, brain cancer, breast cancer, prostate cancer, colorectal cancer, and combinations thereof. In some embodiments, the targeted tumor cells may be associated with brain tumors. In some embodiments, the targeted tumor cells may include cancer stem cells.

### BRIEF DESCRIPTION OF FIGURES

FIG. 1 provides images indicating that cultured primary human glioblastoma multiforme (GBM) cells have an array of active xenobiotic pumps that are capable of exporting various dyes, including Rhodamine 123 (Rh123), carboxy-2′,7′-dichlorofluorescein (BCECF-AM), Hoechst33342, and carboxy-2′,7′-dichlorofluorescein-acetate ester (DCFDA-AM).

FIG. 2 shows data relating to the potentiation of vinblastine (Vin), docetaxel (Doc), and SN-38 toxicity in hydrophilic carbon cluster (HCC) antibody drug enhancement systems (HADES). Vin, Doc and SN-38 were combined with xenobiotic pump inhibitors Haloperidol (Halo) or Indomethicin (Indo). The compositions were then delivered to GBMs in polyethylene glycol hydrophilic carbon clusters (PEG-HCCs) that were associated with anti-IL-13R IgGs. FIG. 2A shows that there is a synergistic effect in dye accumulation using Vin and Doc and either of the xenobiotic pump inhibitors (Halo or Indo). FIG. 2B shows that the levels of living cells falls more than about 50% when cells are treated with Vin or Doc in the presence of Halo. Cell numbers were counted using the center field of n=5 wells. FIG. 2C shows that the dead cell numbers are elevated when cells are treated with Vin or Doc in the presence of Halo. FIGS. 2D-2E shows additional data relating to Halo-mediated potentiation of Vin, Doc and SN38 toxicity in HADES compositions. FIG. 2F shows data relating to drug pump inhibition as a function of dye retention in GBM cells. PEG-HCCs were loaded with Halo, Sulfinpyrazone (Sulf) or Indo. The constructs were then targeted to GBM cells by IL-13R IgGs that were bound to the PEG-HCCs.

FIG. 3 provides data summarizing the potentiation of Vin or Doc toxicities in GBM cells with Halo or Indo via IgG/PEG-HCC delivery. FIG. 3A shows that growing GBM cells for 24 hours in Indo/PEG-HCC (in the presence or absence of antibody targeting) causes a small drop in cell numbers that was statistically insignificant from growth in the presence of Halo/PEG-HCC (in the presence or absence of antibody targeting), White bars represent saline controls. Blue bars represent Doc as IL13R_{AB}/Peg-HCC. Red bars represent Vin as IL13R_{AB}/Peg-HCC. FIG. 3B shows a modified version of the data in FIG. 3A, where only the potentiating effect is shown. The data compares targeted and untargeted xenobiotic pump inhibitors so that the cell numbers in the presence of untargeted pump inhibitor are averaged to 100%. The results show that pump inhibition by Halo increases the toxicity of both Doc and Vin by approximately 50%. In contrast, Indo preferentially increases Vin toxicity by 70% and Doc toxicity by 40%.

FIG. 4 shows that Halo and Indo potentiate the actions of both Vin and Doc in both cervical cancer cells (FIG. 4A) and breast cancer cells (FIG. 4B). The dye retentions for these cells are shown in FIGS. 4C-4D.

FIG. 5 provides schemes for making various HADES compositions. FIGS. 5A-B show coupling of Azido-PEG-Amine to HCC/biotin to generate N₃-PEG-HCC/N₃-PEG-HCC-Biotin, typically using carbodiimide coupling. FIGS. 5C-D show the click coupling of N₃-PEG-HCC/Biotin to surface receptor substrates or peptides.

FIG. 6 provides additional schemes for making various HADES compositions. FIG. 6A shows the coupling of EGFR antagonist Erlotinib to Azido-PEG-HCC/Biotin via click chemistry. FIG. 6B shows the structure of CUDC-101 with ethyne groups that can be used to generate potent multi-targeted HADES compositions via click chemistry. FIG. 6C shows how a membrane androgen receptor can be ligated with Ethisterone (left panel) to treat therapy-resistant prostate cancer, and Ethinylestradiol (right panel) to treat breast cancer or colorectal carcinoma.

FIG. 7 illustrates a scheme for making peptidyl-PEG-HCCs through click chemistry.

FIG. 8 illustrates a scheme for making peptidyl-PEG-Biotin through click chemistry.

FIG. 9 illustrates a scheme for making a click chemistry positive hyaluronic acid.

FIG. 10 provides images illustrating that biotin-PEG-peptide molecules bind to GBM cells (i.e., biopsy samples from BT111 cells).

FIG. 11 provides additional images illustrating that biotin-PEG-peptide molecules bind to the surfaces of GBM cells (i.e., biopsy samples from BT111 cells).

FIG. 12 provides data indicating that peptidyl-PEG-HCCs can be utilized as HADES compositions. For instance, FIG. 12A provides a chart indicating that peptidyl-PEG-HCCs loaded with Vin or Doc can target GBM cells (i.e., BT111 cells). FIGS. 12B-12C provide data illustrating that drug pump inhibitors Halo and Indo potentiate the effects of Vin and Doc on GBM cells.

FIG. 13 provides images indicating that HADES compositions containing Vin, Doc, Halo and Indo can be used to treat breast cancer in a nude mouse model.

FIG. 14 provides an overview of a personalized medicine approach where GBM cells in a brain cancer patient are screened for susceptibility to various HADES compositions.

FIG. 15 provides a mechanism by which HADES compositions can treat cancer. FIG. 15A shows that the addition of HADES compositions to the blood stream can allow for the compositions to target the cancer. FIG. 15B shows that, after treatment, HADES compositions deliver both chemotherapeutic drugs and drug pump inhibitors to cancer cells. Cancers cells that bind chemotherapeutic drugs and drug pump inhibitors (and some nearby neighbor cells) begin to die, thereby releasing cell contents and membrane fragments. These death markers stimulate the immune system to infiltrate the tumor body.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Cancer is a leading cause of death worldwide, accounting for 13% of all deaths, including almost 560,000 Americans yearly. Many chemotherapeutic drugs have been developed, the majority of which are highly toxic to rapidly proliferating cells. However, in many cases, these chemotherapeutic drugs have failed due to a combination of factors.

In particular, there are two major reasons for the failure of chemotherapeutic cancer drugs. First, the chemotherapeutic composition is usually given at a low dosage in order to avoid the widespread death of normal but highly proliferating cells types (e.g., cells of the intestinal lining and immune cells). Second, many cancer cells acquire chemotherapy resistance. Without being bound by theory, it is envisioned that chemotherapy resistance can be due to the up-regulation of a range of xenobiotic cell membrane pumps. For instance, Table 1 shows the five major drug transporters that are instrumental in bestowing drug resistance to chemotherapeutic compounds, including P-glycoprotein (P-gp), Breast cancer resistance protein (BCRP), and multi-drug resistance proteins-1, -2 and -7 (MRP1, MRP2 and MRP7, respectively).

| | Affected Drugs | Dyes Pumped by | Inhibitor (I) |
|---|---|---|---|
| Transporter | (Resistance) | Transporter | or Substrate (S) |
| MDR1 | Vinblastine | Hoechst 33342 | (I) Haloperidol |
| (P-glyco- | Docetaxel | Rhodamine 123 | |
| protein P-gp) | SN-38 | | |
| Breast cancer | SN-38 | Hoechst33342 | (S) Indomethacin |
| resistance | Mitoxantrone | Rhodamine 123 | (I) Fumitremorgin C |
| protein | Daunorubicin | | |
| (BCRP, | Doxorubicin | | |
| ABCG2) | Topotecan | | |
| | Epirubicin | | |
| MRP1 | Vinblastine | Rhodamine 123 | Sulfinpyrazone |
| | SN-38 | BCECF | Indomethacin |
| MRP2 | Vinblastine | BCECF | Sulfinpyrazone |
| | | | Indomethacin |
| MRP7 | Vincristine | Rhodamine 123 | Sulfinpyrazone |
| | Vinblastine | | Neratinib |
| | Paclitaxel | | |
| | Docetaxel | | |
| | Paclitaxel | | |
| | Docetaxel | | |
| | Vincristine | | |
| | Etoposide | | |
| | SN-38 | | |
| | Daunorubicin | | |

Table 1 provides a summary of transporters that are up-related in cancer, their corresponding resistand drugs, the dyes pumped by the transporters, and their inhibitors or substrates.

For instance, in primary glioma, there is considerable heterogeneity in the levels of different pumps in tumors. Furthermore, part of chemotherapeutic resistance may not only be a function of the presence of drug pumping activity of the cancer cells themselves, but of pumps present in the endothelial cells that feed the tumor. In particular, endothelial cells that supply a glioma have been shown to have aberrant surface protein expression, including the presence of 4F2 heavy chain antigen and Prostate Specific Membrane Antigen (PSMA).

The aforementioned obstacles to the efficacy of chemotherapeutic cancer drugs are further escalated when desired drugs are hydrophobic. Additional obstacles include lack of effective methods of making personalized drug delivery compositions that effectively target a desired tumor in a particular subject. Therefore, more efficient and effective approaches to targeted drug delivery are desired for treating various types of tumors. The methods and therapeutic compositions of the present disclosure address the aforementioned limitations.

In particular, various embodiments of the present disclosure pertain to therapeutic compositions for specifically targeting tumor cells. Further embodiments of the present disclosure pertain to methods of targeting tumor cells in a subject by administering the therapeutic compositions of the present disclosure to the subject. Additional embodiments of the present disclosure pertain to personalized methods of formulating therapeutic compositions for targeting tumor cells in a particular subject.

Therapeutic Compositions

Various embodiments of the present disclosure pertain to therapeutic compositions for targeting one or more tumor cells, such as brain tumor cells. In some embodiments, the therapeutic compositions of the present disclosure generally include: (1) a plurality of nanovectors; (2) one or more active agents associated with the nanovectors, where the one or more active agents have activity against the tumor cells; (3) one or more active agent enhancers associated with the nanovectors; and (4) one or more targeting agents associated with the nanovectors, where the one or more targeting agents have recognition activity for one or more markers of the tumor cells.

The therapeutic compositions of the present disclosure can have numerous variations. For instance, in some embodiments, the one or more active agents and the one or more active agent enhancers are associated with the same nanovector molecules. In other embodiments, the one or more active agents and the one or more active agent enhancers are associated with different nanovector molecules. For instance, in some embodiments, one or more active agents are associated with a first nanovector molecule that is associated with a first targeting agent. Likewise, one or more active agent enhancers are associated with a second nanovector molecule that is associated with a second targeting agent. Additional variations can also be envisioned. Furthermore, as set forth in more detail below, various nanovectors, active agents, targeting agents, and active agent enhancers may be utilized in the therapeutic compositions of the present disclosure.

Nanovectors

Nanovectors suitable for use in the therapeutic compositions of the present disclosure generally refer to particles that are capable of associating with active agents, active agent enhancers, and targeting agents. Nanovectors in the present disclosure also refer to particles that are capable of delivering one or more active agents and active agent enhancers to a targeted area.

In some embodiments, suitable nanovectors include, without limitation, single-walled carbon nanotubes (SWNTs), double-walled nanotubes (DWNTs), triple-walled nanotubes (TWNTs), multi-walled nanotubes (MWNTs), ultra-short nanotubes, ultra-short single-walled carbon nanotubes (US-SWNTs), hydrophilic carbon clusters (HCCs), graphene nanoribbons, graphite, graphite oxide nanoribbons, graphene quantum dots, carbon black, derivatives thereof, and combinations thereof.

In some embodiments, the nanovectors of the present disclosure may be modified in various ways. For instance, in some embodiments, the nanovectors of the present disclosure may be oxidized. In some embodiments, the nanovectors of the present disclosure may be functionalized with one or more molecules, polymers, chemical moieties, solubilizing groups, functional groups, and combinations thereof. For instance, in some embodiments, the nanovectors of the present disclosure may be functionalized with ketones, alcohols, epoxides, carboxylic acids, and combinations thereof.

In more specific embodiments, the nanovectors of the present disclosure may be functionalized with a plurality of solubilizing groups. In further embodiments, the solubilizing groups may include at least one of polyethylene glycols (PEGs), polypropylene glycol (PPG), poly(p-phenylene oxide) (PPOs), polyethylene imines (PEI), poly(vinyl alcohol) (PVA), poly(acrylic acid) (PAA), poly(vinyl amines), and combinations thereof. In more specific embodiments, the nanovectors of the present disclosure can include PEG-functionalized HCCs (i.e., PEG-HCCs, as described in more detail below).

The nanovectors of the present disclosure may also have various properties. For instance, in some embodiments, the nanovector may be hydrophilic (i.e., water soluble). In some embodiments, the nanovectors of the present disclosure may have both hydrophilic portions and hydrophobic portions. For instance, in some embodiments, the nanovectors of the present disclosure may have a hydrophilic domain (e.g, a hydrophilic surface) and a hydrophobic domain (e.g., a hydrophobic cavity). The nanovectors of the present disclosure can also be engineered to possess both hydrophobic and hydrophilic domains, combining high aqueous solubility with the ability to adsorb hydrophobic compounds. In some embodiments, this duality of hydrophilic and hydrophobic domains can result in the formation of structures resembling micelles or liposomes. Such structures can in turn further entrap active agents for delivery to a desired site.

In further embodiments, the therapeutic compositions of the present disclosure may have hydrophobic domains and hydrophilic domains. In further embodiments, the one or more active agents and active agent enhancers are associated with the hydrophobic domains, and the one or more targeting agents are associated with the hydrophilic domains.

In more specific embodiments, the nanovectors of the present disclosure include US-SWNTs. US-SWNTs are also referred to as hydrophilic carbon clusters (HCCs). Therefore, for the purposes of the present disclosure, US-SWNTs are synonymous with HCCs. In some embodiments, HCCs can include oxidized carbon nanoparticles that are about 30 nm to about 40 nm long, and approximately 1-3 nm wide.

In some embodiments, US-SWNTs (i.e., HCCs) may be produced by reacting SWNTs in fuming sulfuric acid with nitric acid to produce a shortened carbon nanotube characterized by opening of the nanotube ends. Such methods are disclosed in Applicants' co-pending U.S. patent application Ser. No. 12/280,523, entitled “Short Functionalized, Soluble Carbon Nanotubes, Methods of Making Same, and Polymer Composites Made Therefrom.” This may be followed by the functionalization of the plurality of carboxylic acid groups. In some embodiments, the HCC may be an oxidized graphene.

In some embodiments, the HCCs may be functionalized with one or more solubilizing groups, such as PEGs, PPGs, PPOs, PEIs, PVAs, PAAs, poly(vinyl amines), and combinations thereof (as previously described). In more specific embodiments, the nanovectors of the present disclosure may include polyethylene glycol-functionalized HCCs (PEG-HCCs). Various PEG-HCCs and methods of making them are disclosed in the following articles and applications: Berlin et al., ACS Nano 2010, 4, 4621-4636; Lucente-Schultz et al., J. Am. Chem. Soc. 2009, 131, 3934-3941; Chen et al., J. Am. Chem. Soc. 2006, 128, 10568-10571; Stephenson, et al., Chem. Mater. 2007, 19, 3491-3498; Price et al., Chem. Mater. 2009, 21, 3917-3923; PCT/US2008/078776; and PCT/US2010/054321.

In various embodiments, PEG-HCCs (and other functionalized forms of HCCs) may have various advantageous properties for use as nanovectors. For instance, PEG-HCCs (and other functionalized forms of HCCs) may demonstrate low biological toxicity with clearance mainly through the kidneys. PEG-HCCs (and other functionalized forms of HCCs) may also contain hydrophobic domains that can be non-covalently loaded with active agents, such as hydrophobic active agents. In addition, PEG-HCCs (and other functionalized forms of HCCs) can have an ability to strongly bind to various targeting agents (such as peptides or antibodies) without significantly interfering with the activity of the targeting agents. Thus, active agent-loaded PEG-HCCs (and other functionalized forms of HCCs) combined with a targeting agent can be used to bind to a chosen cell surface antigen and deliver a hydrophobic, lipophilic active agent into or on cells that express a selected epitope.

Other suitable PEGylated or functionalized carbon nanomaterials can also be used as nanovectors. Non-limiting examples include PEGylated graphite oxide nanoribbons (PEG-GONR), PEGylated oxidized carbon black (PEG-OCB), and PEGylated carbon black (PEG-CB). Additional suitable nanovectors, including PEG-HCCs, are disclosed in U.S. patent application Ser. No. 12/245,438; PCT/US2008/078776; and PCT/US2010/054321. The use of other suitable nanovectors not disclosed here can also be envisioned.

Active Agents

Active agents of the present disclosure generally refer to biologically active compounds, such as compounds that have activity against various tumor cells, such as brain tumor cells (e.g., anti-apoptoic activity, anti-proliferative activity, anti-oxidative activity, etc.). For instance, in various embodiments, active agents of the present disclosure may refer to anti-cancer drugs, chemotherapeutics, antioxidants, and anti-inflammatory drugs. Furthermore, the active agents of the present disclosure may be derived from various compounds. For instance, in various embodiments, the active agents of the present disclosure can include, without limitation, small molecules, proteins, peptides, aptamers, DNA, anti-sense oligo nucleotides, miRNA, siRNA, and combinations thereof.

In more specific embodiments, the active agents of the present disclosure may be at least one of cis-platin, SN-38, vinblastine, daunorubicin, paclitaxel, docetaxel, doxorubicin, epirubicin, vincristine, iadarubicin, mitoxantrone, oxaliplatin, topotecan, etoposide, erlotinib, ethisterone, ethinylestradiol, 1,2,3,4-tetrahydronaphthalene-2,3-diamine, 2,2-dichloro-octahydrocyclohexa 1,3-diaza-2-platinacyclopentane, 2,2-dichloro-hexahydro-naphtho1,3-diaza-2-platinacyclopentane, 4,4-dichloro-3,5-diaza-4-platinatetracycloheptadecahexaene, nitrogen mustards, spermine mustards, estrogen mustards, cholesterol mustards, combinations thereof, and derivatives thereof.

Furthermore, the active agents of the present disclosure may have various properties. For instance, in some embodiments, the active agents may be hydrophobic. In fact, an advantage of the present disclosure is the effective delivery of hydrophobic active agents that may have been otherwise insoluble. As set forth in more detail below, such hydrophobic agents can be associated with various nanovectors for direct delivery to a desired tumor site without requiring the use of moieties that increase solubility but limit active agent efficacy.

The active agents of the present disclosure may also be associated with nanovectors in various manners. For instance, in some embodiments, the active agents may be non-covalently associated with nanovectors, such as through sequestration, adsorption, ionic bonding, dipole-dipole interactions, hydrogen bonding, Van der Waals interactions, and other types of non-covalent associations.

In some embodiments, the active agents may be non-covalently sequestered within a cavity, domain or surface of a nanovector. In some embodiments, the active agents may be sequestered from their surrounding environment by non-covalent association with a nanovector's solubilizing groups. In more specific embodiments where the nanovector includes hydrophobic domains and hydrophilic domains, the active agent may be associated with a hydrophobic domain. In further embodiments, a hydrophobic active agent may be associated with a hydrophobic domain of a nanovector. In some embodiments, this duality of hydrophilic and hydrophobic domains can result in the formation of structures resembling micelles or liposomes that can further entrap the active agents for delivery.

In further embodiments, the active agents of the present disclosure may be covalently associated with nanovectors. For instance, in some embodiments, the active agents of the present disclosure may be covalently associated with an active agent through a linker molecule, through a chemical moiety, or through a direct chemical bond between the active agent and the nanovector. In some embodiments, the active agent may be covalently associated with the nanovector through a cleavable moiety, such as an ester bond or amide bond. In some embodiments, the cleavable moiety may be a photo-cleavable moiety or a pH sensitive cleavable moiety. Additional modes by which active agents may be covalently or non-covalently associated with nanovectors can also be envisioned.

In some embodiments, the therapeutic compositions of the present disclosure may include a single active agent. In some embodiments, therapeutic compositions of the present disclosure may include multiple active agents. In further embodiments set forth below, the therapeutic compositions of the present disclosure may also include one or more enhancers of active agents.

Enhancers of Active Agents

Enhancers of active agents generally refer to any compounds or molecules that enhance the activity of active agents. In some embodiments, the active agent enhancers include drug transport pump inhibitors, such as xenobiotic pump inhibitors. In some embodiments, the drug transport pump inhibitors inhibit the activity of ABC transporters, such as ABCB1, ABCC1, ABCC2, ABCC3, ABCC4, ABCG2, and combinations thereof. In some embodiments, the active agent enhancers may include at least one of fumitremorgan C, indomethacin, 6-thioguanine, sulfate, guggulsterone, tolmetin, haloperidol, sulfinpyrazone, chrysin, gleevec, neratinib, and combinations thereof. Without being bound by theory, it is envisioned that the use of such drug transport pump inhibitors will prevent the pumping of active agents out of cells, thereby enhancing their activity within cells. Additional examples of drug transport pump inhibitors are set forth in the Examples below.

The active agent enhancers of the present disclosure may also be associated with nanovectors in various manners. For instance, in some embodiments, the active agent enhancers may be non-covalently associated with nanovectors, such as through sequestration, adsorption, ionic bonding, dipole-dipole interactions, hydrogen bonding, Van der Waals interactions, and other types of non-covalent associations.

In some embodiments, the active agent enhancers may be non-covalently sequestered within a cavity, domain or surface of a nanovector. In some embodiments, the active agent enhancers may be sequestered from their surrounding environment by non-covalent association with a nanovector's solubilizing groups. In more specific embodiments where the nanovector includes hydrophobic domains and hydrophilic domains, the active agent enhancers may be associated with a hydrophobic domain. In further embodiments, a hydrophobic active agent enhancer may be associated with a hydrophobic domain of a nanovector. In some embodiments, this duality of hydrophilic and hydrophobic domains can result in the formation of structures resembling micelles or liposomes that can further entrap the active agent enhancers for delivery.

In further embodiments, the active agent enhancers of the present disclosure may be covalently associated with nanovectors. For instance, in some embodiments, the active agent enhancers of the present disclosure may be covalently associated with a nanovector through a linker molecule, through a chemical moiety, or through a direct chemical bond between the active agent and the nanovector. In some embodiments, the active agent enhancers may be covalently associated with the nanovector through a cleavable moiety, such as an ester bond or amide bond. In some embodiments, the cleavable moiety may be a photo-cleavable moiety or a pH sensitive cleavable moiety. Additional modes by which active agent enhancers may be covalently or non-covalently associated with nanovectors can also be envisioned.

In some embodiments, the therapeutic compositions of the present disclosure may include a single active agent enhancer. In some embodiments, the therapeutic compositions of the present disclosure may include multiple active agent enhancers. In some embodiments, the active agent enhancers of the present disclosure may be associated with the same nanovector molecules that are associated with active agents. In some embodiments, the active agent enhancers of the present disclosure may be associated with different nanovector molecules that are not associated with active agents.

Tracers

The therapeutic compositions of the present disclosure can also be associated with one or more tracers, such as an MRI tracer. In more specific embodiments, the tracer(s) associated with therapeutic compositions may include a gadolinium tracer, such as Gd3⁺. In further embodiments, the tracer may include, without limitation, at least one of fluorescent molecules, Qdots, radioisotopes, and combinations thereof. In various embodiments, such tracers can be used to track in real-time the location, distribution and delivery of administered therapeutic compositions. Thus, such embodiments would allow a physician to follow the degree of therapeutic composition binding to tumors, monitor the biological half-life of the therapeutic compositions, and monitor accumulation in non-target organs, such as the kidney and liver.

Targeting Agents

Targeting agents of the present disclosure generally refer to compounds that target a particular marker, such as markers associated with tumor cells. In various embodiments, the targeting agents may include, without limitation, antibodies, RNA, DNA, aptamers, small molecules, dendrimers, proteins, peptides and combinations thereof. In more specific embodiments, the targeting agents may include peptides. In particular embodiments, the peptides may include synthetic peptides, such as synthetic peptides selected from a phage display library. In more specific embodiments, the targeting agent is a peptide directed against a cell surface receptor that is up-regulated in tumor cells. See, e.g., Table 2 in Example 3.

In some embodiments, the targeting agents may include peptides that specifically target epidermal growth factor receptors. As set forth in more detail below, epidermal growth factor receptors (EGFRs) are over-expressed in many types of cancer cell lines. Thus, peptides that bind to EGFRs may be used to deliver active agents and active agent enhancers to the cancer cells in various embodiments.

In some embodiments, the targeting agents may include small molecules directed against a marker of tumor cells. In more specific embodiments, the small molecule may include hyaluronates, such as hyaluronic acid.

Targeting agents may be associated with nanovectors in various manners. In some embodiments, targeting agents may be non-covalently associated with nanovectors, such as through sequestration, adsorption, ionic bonding, dipole-dipole interactions, hydrogen bonding, Van der Waals interactions, and other types of non-covalent associations.

In more specific embodiments, targeting agents may be non-covalently sequestered on a surface of a nanovector. In some embodiments, targeting agents may be covalently associated with nanovectors. In some embodiments, targeting agents may be covalently and non-covalently associated with nanovectors.

In more specific embodiments, the targeting agents of the present disclosure may be covalently associated with nanovectors through a linker molecule, through a chemical moiety, or through a direct chemical bond between the targeting agent and the nanovector. In some embodiments, the targeting agent may be covalently associated with the nanovector through a cleavable moiety, such as an ester bond or amide bond. In some embodiments, the cleavable moiety may be a photo-cleavable moiety or a pH sensitive cleavable moiety. Additional modes by which targeting agents may be covalently or non-covalently associated with nanovectors can also be envisioned.

Markers

As set forth previously, targeting agents of the present disclosure can target various markers associated with tumor cells. In some embodiments, such markers may be on a surface of tumor cells. In some embodiments, such markers may be within tumors cells. In some embodiments, such markers can include epitopes associated with tumor cells. In some embodiments, such epitopes may be over-expressed or up-regulated in tumor cells relative to other cell types.

In some embodiments, the marker is a receptor on a surface of tumor cells. Examples of such receptors include, without limitation, epidermal growth factor receptors, cytokine receptors, interleukin receptors, interleukin-13 receptors, interleukin-4 receptors, transferrin receptors, neuropilin receptors, vascular endothelial growth factor receptors, integrins, gastrin-releasing peptide receptors, hepatocyte growth factor receptors, HER-2 receptors, prostate specific membrane antigens, c-met, and combinations thereof. In further embodiments, the marker is interleukin-13 receptor (IL-13R), a cytokine receptor that is up-regulated in a large range of brain tumors, including glioblastoma multiformes (GBMs). In more specific embodiments, the marker is the epidermal growth factor receptor (EGFR), a receptor over-expressed, in either full length or truncated form, in many cancers, including GBMs. Additional markers can also be envisioned as suitable targets for various tumor cells.

Tumor Cells

The therapeutic compositions of the present disclosure can be used to target various tumor cells. In some embodiments, the tumor cells may include cancer stem cells. In some embodiments, the tumor cells may be associated with at least one of cervical cancer, brain cancer, breast cancer, prostate cancer, colorectal cancer, and combinations thereof. In various embodiments, the cancers may be malignant, benign, primary, or metastatic.

In some embodiments, the tumor cells may be associated with brain tumors. Non-limiting examples of brain tumor types include, without limitation, gliomas, meningiomas, pituitary adenomas, and combinations thereof. Non-limiting examples of gliomas include ependymomas, astrocytomas, oligodendrogliomas, mixed gliomas (e.g., oligoastrocytomas), and combinations thereof. More specific examples of tumors that can be targeted by the therapeutic compositions of the present disclosure may include, without limitation, gliomas, glioblastomas, astrocytomas, neuroblastomas, retinoblastomas, meduloblastomas, oligodendrogliomas, ependymomas, choroid plexus papillomas, and combinations thereof. In more specific embodiments, the brain tumor to be targeted is a primary glioblastoma multiforme (GBM).

In various embodiments, the targeted brain tumors may be malignant, benign, primary, or metastatic. In some embodiments, the targeted brain tumors may be located in different parts of the brain. In some embodiments, the targeted brain tumors may have spread to different parts of the body.

Methods of Targeting Tumor Cells

Further embodiments of the present disclosure pertain to methods of targeting tumor cells (e.g., brain tumors) in a subject. Such methods generally include administering one or more of the above-described therapeutic compositions to the subject.

Subjects

The therapeutic compositions of the present disclosure may be administered to various subjects. In some embodiments, the subject is a human being. In some embodiments, the subject is a human being with a brain tumor, such as a glioma. In some embodiments, the subjects may be non-human animals, such as mice, rats, other rodents, or larger mammals, such as dogs, monkeys, pigs, cattle and horses.

Modes of Administration

The therapeutic compositions of the present disclosure can be administered to subjects by various methods. For instance, the therapeutic compositions of the present disclosure can be administered by oral administration (including gavage), inhalation, subcutaneous administration (sub-q), intravenous administration (I.V.), intraperitoneal administration (I.P.), intramuscular administration (I.M.), intrathecal injection, and combinations of such modes. In further embodiments of the present disclosure, the therapeutic compositions of the present disclosure can be administered by topical application (e.g, transderm, ointments, creams, salves, eye drops, and the like). Additional modes of administration can also be envisioned.

Variations

In various embodiments, the therapeutic compositions of the present disclosure may be co-administered with other therapies. For instance, in some embodiments, the therapeutic compositions of the present disclosure may be co-administered along with other anti-cancer drugs. In some embodiments, the therapeutic compositions of the present disclosure may be administered to patients undergoing chemotherapy. Other modes of co-administration can also be envisioned.

Personalized Methods of Formulating Therapeutic Compositions

Additional embodiments of the present disclosure pertain to personalized methods of formulating therapeutic compositions. Such methods generally include one or more of the following steps: (1) isolating tumor cells from a subject; (2) determining the susceptibility of the tumor cells to one or more active agents; (3) determining expression levels of one or more markers of the tumor cells; and (4) formulating therapeutic compositions based on one or more of the aforementioned steps. In some embodiments, the susceptibility of the tumor cells to one or more active agents may be determined in the presence of one or more active agent enhancers.

For instance, a formulated therapeutic composition may include one or more active agents and active agent enhancers that were selected based on the determined susceptibility of the tumor cells to the active agent(s) in the presence of the active agent enhancer(s). Likewise, a formulated therapeutic composition may include one or more targeting agents that have recognition activities for one or more markers of tumor cells that were selected based on the determined expression levels of the marker(s). Advantageously, such tailored methods allow for the formulation of therapeutic compositions that can specifically target tumor cells with a specified epitopic landscape for active agent delivery.

The aforementioned tailored methods of formulating therapeutic compositions have additional variations. For instance, in some embodiments, the methods may only include a step of determining expression levels of one or more markers of the tumor cells and formulating therapeutic compositions based on such determinations. Likewise, in other embodiments, the methods may include only a step of determining susceptibility of the tumor cells to one or more active agents and formulating therapeutic compositions based on such determinations. In other embodiments, the methods may include steps of determining expression levels of one or more markers of the tumor cells, determining susceptibility of the tumor cells to one or more active agents, and formulating therapeutic compositions based on such determinations.

Likewise, various methods may be used to isolate tumor cells from a subject. In some embodiments, the isolation methods may include an excision of a portion of a tumor from the subject. In some embodiments, standard biopsy techniques may be utilized to make such excisions.

Various methods may also be used to determine the susceptibility of tumor cells to one or more active agents. For instance, in some embodiments, the susceptibility is determined by growing different batches of the tumor cells in the presence of different active agents and comparing the growth rates of the different batches with the growth rate of untreated brain tumor cells. Standard tissue culture techniques may be used for such methods. In some embodiments, one or more of the active agents that confer the slowest growth rate on tumor cells may be selected for incorporation into therapeutic compositions. In various embodiments, the aforementioned methods may occur in the presence or absence of one or more active agent enhancers.

Various methods may also be used to determine the expression levels of one or more markers of the tumor cells. For instance, in some embodiments, the expression levels of one or more markers may be determined by treating the tumor cells with targeting agents that are specific for the markers. In various embodiments, standard epitope mapping techniques may be utilized for determining such expression levels. In some embodiments, the markers may be epitopes, receptors, or proteins that are over-expressed or up-regulated on the surface of tumor cells relative to other cells (e.g., IL-13R, GFAP, EGFR, etc.). In some embodiments, targeting agents that are selected for incorporation into therapeutic compositions may be specific for such over-expressed markers.

The personalized methods of formulating therapeutic compositions in the present disclosure may be tailored towards various subjects. In some embodiments, the subject is a human being. In some embodiments, the human being may be suffering from a brain cancer, such as glioblastoma. In further embodiments, the subject may be a non-human animal, as discussed previously.

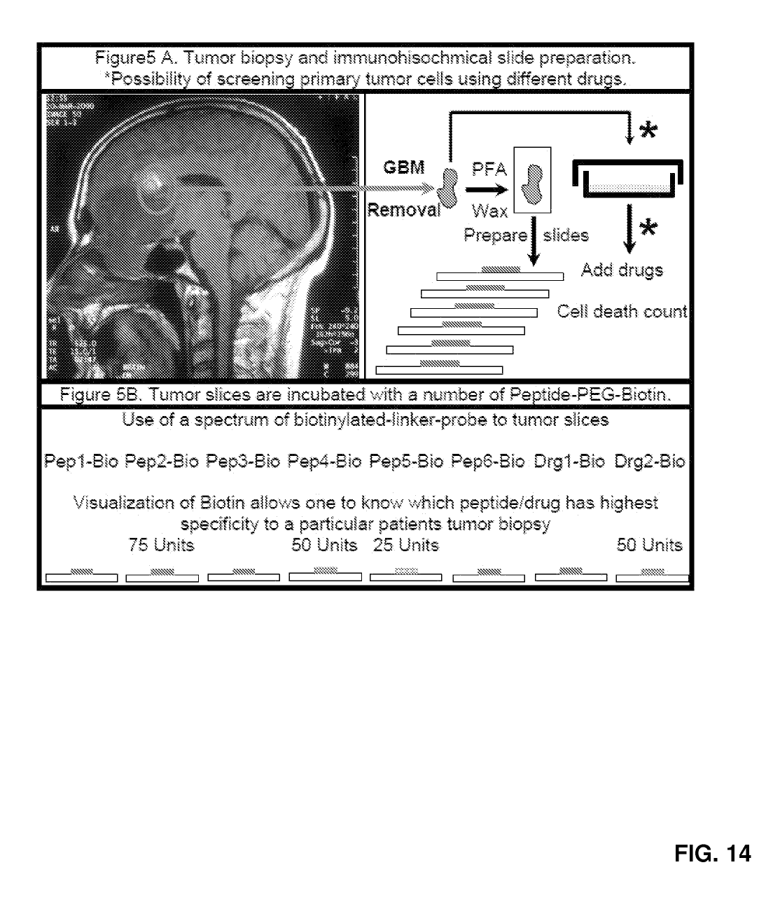

A more specific personalized method of formulating a therapeutic composition is illustrated in FIG. 14. The scheme in FIG. 14 outlines a method of formulating a therapeutic composition to treat a patient with a brain tumor (e.g., GBM). The brain tumor is excised by standard biopsy procedures. After excision, part of the tumor is fixed, waxed, sliced, mounted, dewaxed, and rehydrated. Part of the excised tumor can also be grown in tissue culture in order to identify the chemotherapeutic drugs to which the individual tumor is most susceptible. Next, the treated tumor slices undergo peptide-based screening to identify the levels of tumor-specific surface antigens in the individual tumor. Thereafter, the information obtained can be used to formulate specific therapeutic agents. Targeting agents of choice (e.g., peptides) are then mixed with nanovectors (e.g., PEG-HCCs) that have been pre-loaded with active agents and active agent enhancers. Using this methodology, a large number of different active agent-loaded nanovectors can be manufactured and stored. A physician can then make an informed choice as to which active agents, active agent enhancers, and targeting agents to use for a particular subject based on the attributes of the subject's tumor (e.g., expression levels of different markers and the susceptibility of tumors to various active agents).

Formulating Therapeutic Compositions

Various methods may also be used to formulate the therapeutic compositions of the present disclosure. Such methods generally include: (1) associating nanovectors with one or more active agents and active agent enhancers; and (2) associating one or more targeting agents with the nanovectors. In some embodiments, one or more of the above-mentioned associations may occur non-covalently, such as by sequestration, adsorption, ionic bonding, dipole-dipole interactions, hydrogen bonding, Van der Waals interactions, and other types of non-covalent interactions. In further embodiments, one or more of the associations may occur by covalent bonding.

In various embodiments, the aforementioned associations may occur simultaneously or sequentially. In some embodiments, the associations may occur by mixing a nanovector with one or more active agents, active agent enhancers, and targeting agents. In some embodiments, a first batch of nanovectors may be mixed with one or more active agents and one or more targeting agents. A second batch of the nanovectors may then be mixed with one or more active agent enhancers and one or more targeting agents. The first and the second batches may then be mixed together.

Therapeutic compositions of the present disclosure can also be formulated in conventional manners. In some embodiments, the formulation may also utilize one or more physiologically acceptable carriers or excipients. The pharmaceutical compositions can also include formulation materials for modifying, maintaining, or preserving various conditions, including pH, osmolarity, viscosity, clarity, color, isotonicity, odor, sterility, stability, rate of dissolution or release, and/or adsorption or penetration of the composition. Suitable formulation materials include, but are not limited to: amino acids (e.g., glycine); antimicrobials; antioxidants (e.g., ascorbic acid); buffers (e.g., Tris-HCl); bulking agents (e.g., mannitol and glycine); chelating agents (e.g., EDTA); complexing agents (e.g., hydroxypropyl-beta-cyclodextrin); and the like. Additional methods of formulating therapeutic compositions can also be envisioned.

Advantages

In some embodiments, the present disclosure can address two major problems with chemotherapy. First, the methods and compositions of the present disclosure can specifically target cancer cells with chemotherapeutics by increasing the local concentration of these drugs in the tumor, as compared to the body's other tissues. Secondly, by subjecting the cancer cells to co-therapy with drug pump inhibitors, the methods and compositions of the present disclosure can inhibit a major method of drug detoxification within cancer cells (i.e., the ability of cancer cells to pump chemotherapeutic compounds from their cytosol or nucleus). Thus, the methods and compositions of the present disclosure can expand the therapeutic window of existing chemotherapeutics and thereby allow patients to receive a much higher dosage of drugs with minimal side-effects that result from chemotherapeutic interactions with normal tissues or cells. Additionally, the methods and compositions of the present disclosure can increase the toxicity of these chemotherapeutics with respect to cancer cells.

### ADDITIONAL EMBODIMENTS

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1

### Potentiation of Cancer Drug Efficacy by Drug Pump Inhibitors

In this Example, Applicants demonstrate that three human cancer types (glioblastoma multiforme or GBM, cervical cancer and breast cancer) can be treated with chemotherapeutics. In this Example, Applicants also demonstrate that the toxicity of the chemotheraputics can be improved by using xenobiotic pump inhibitors, such as Haloperidol (Halo) and Indomethacin (Indo). Applicants also show how these drugs and pump inhibitors can be delivered to the surface of cancer cells using antibody guided, pegylated hydrophobic/hydrophilic carbon clusters (PEG-HCCs).

Primary Human Glioblastomas have Drug Pumps

Applicants investigated the ability of human primary glioma cells to retain dyes in the absence and presence of known drug pump inhibitors. Human GBM cells were grown to confluence and then incubated for ninety minutes with 100 μM Rhodamine 123 (Rh123), 100 μM 2′,7′-bis-(2-carboxyethyl)-5-(and-6)-carboxyfluorescein, acetoxymethyl ester (BCECF-AM), 10 μM Hoechst 33342 (Hoe), and known xenobiotic pump inhibitors in the manner described by Aszalos and Taylor (Methods Mol Biol. 596 (2010) 123-139). After ninety minutes, the cells were fixed using ice-cold 2% paraformaldehyde. The fixed cells were stored overnight in a refrigerator. The cells were then washed three times in phosphate buffered saline at pH 7.4 (PBS) and imaged in a fluorescence microscope.

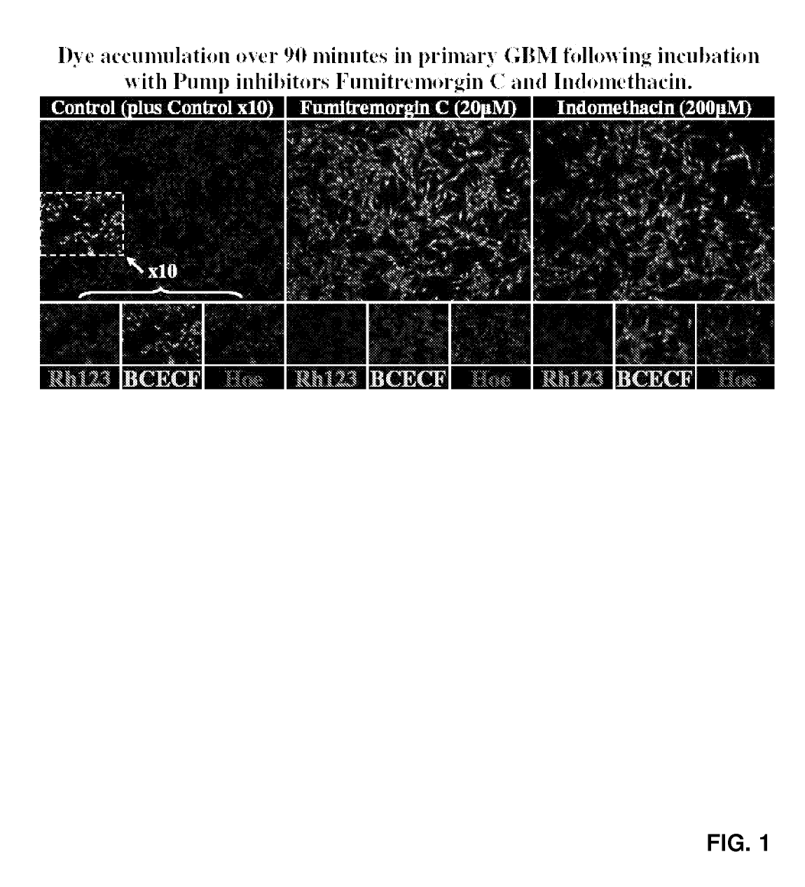

In FIG. 1, Applicants show representative images taken at ×4 magnification showing change in the retention of dyes in the presence of 20 μM Fumitremorgin C, an inhibitor of BCRP, and in the presence of 200 μM Indomethacin (Indo), an inhibitor of BCRP, MDR1 and MDR 2. FIG. 1 demonstrates that the retention of dyes by the uninhibited cancer cells is much lower than it is in the presence of the two inhibitors, and that these pumps are present in a heterogeneous manner, with a few cells becoming very bright in the presence of a single pump inhibitor. The insert in the left panel of FIG. 1 shows the RGB fluorescent levels of control cells multiplied by a factor of 10. This multiplication is desired to achieve similar emission levels to that seen in GBM cells incubated with xenobiotic pump inhibitors. The individual RGB insets at the bottom of the control panel indicate that all three dyes Rh123 (red), BCECF(green) and Hoe(blue) are present in the control cell cytosol at low levels. Fumitremorgin C and Indo both increase dye retention of all dyes, albeit to different extents. Thus, FIG. 1 demonstrates that, as the known drug pump inhibitors alter dye retention, xenobiotic pumps are actively expressed in these human primary GBM cells.

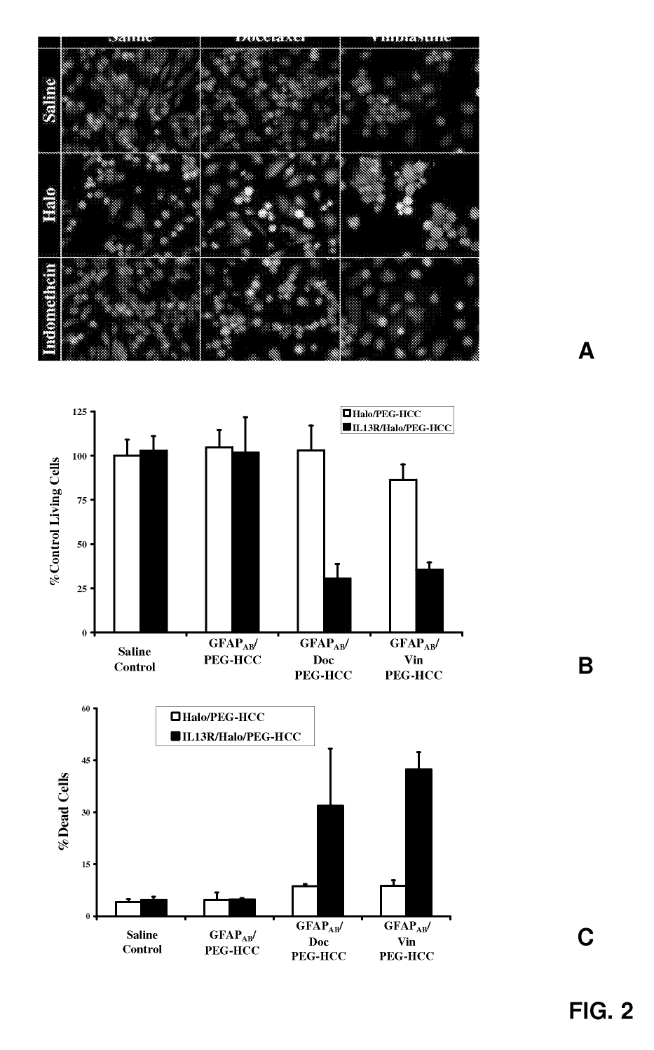

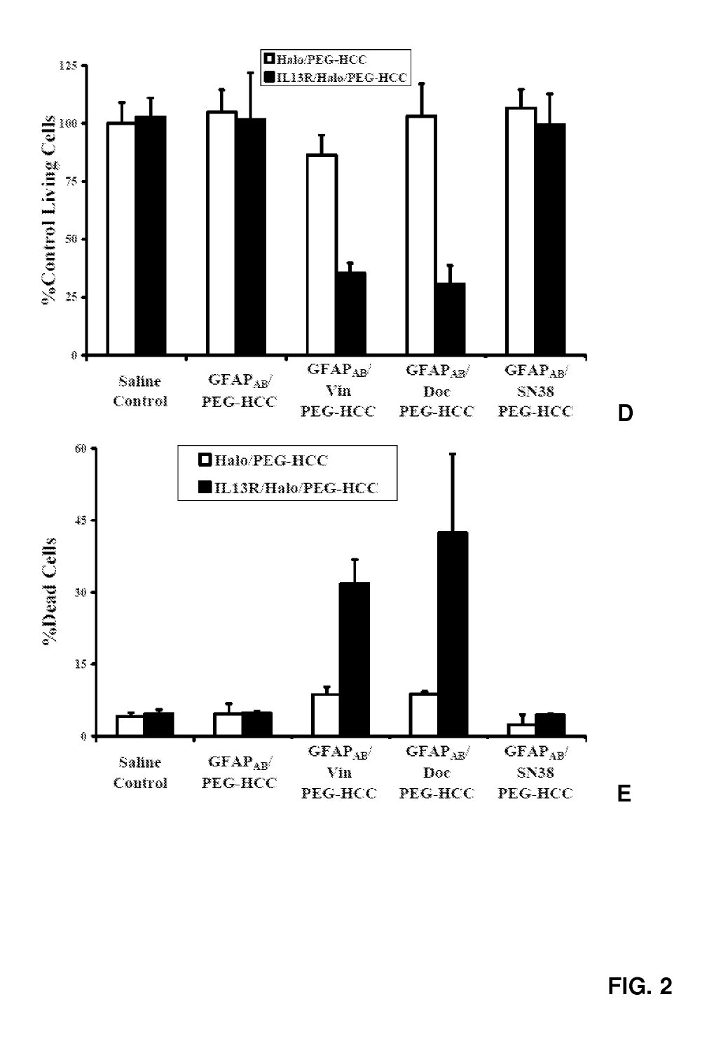

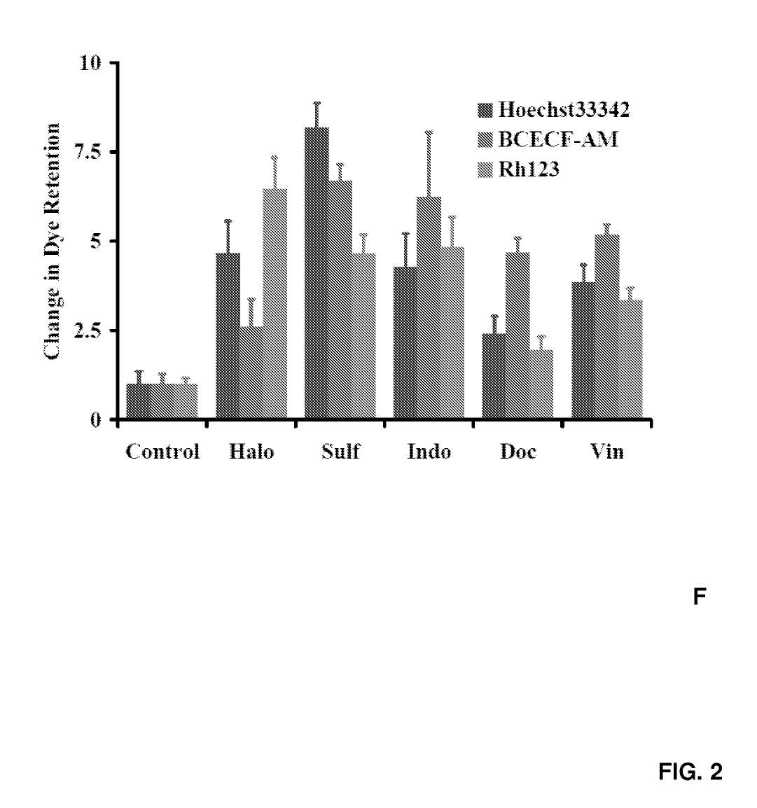

FIG. 2F shows additional data relating to drug pump inhibition as a function of dye retention in GBM cells. In this experiment, PEG-HCCs were loaded with Halo, Sulfinpyrazone (Sulf) or Indo. The same methodologies outlined above were used. The constructs were then targeted to GBM cells by IL-13R IgGs. The same experimental results were obtained.

Drug Pump Inhibition Potentiates Chemotherapeutic Drug Action in GBM

Applicants previously demonstrated the ability to adsorb hydrophobic compounds, such as the chemotherapeutic drug compounds vinblastine (Vin) and docetaxel (Doc), on PEG-HCCs See, e.g., PCT/US2012/35267, PCT/US2010/54321, and PCT/US2008/078776.

Moreover, Applicants have shown the ability to specifically target these nanovectors to the surface of cells by mixing the PEG-HCCs with an antibody that can bind to a cell surface epitope. See, e.g., PCT applications referenced above and ACS Nano 6 (2012) 3114-3120.

In FIG. 2, Applicants demonstrate that chemotherapeutic drugs and pump inhibitors, either singly or together, are able to alter the retention of xenobiotic pump dye substrates in glioblastoma cells grown in culture. The drugs (Vin and Doc) were delivered to a final concentration of 100 nM in the form of GFAP_{AB}/Drug/PEG-HCC. In some experiments, pump inhibitors Haloperidol (Halo) and Indomethacin (Indo) were added at a final concentration of 2 μM in the form of Il-13R_{AB}/Inhibitor/PEG-HCC. Controls consisting of unloaded PEG-HCC, drug/inhibitor loaded PEG-HCC, GFAP_{AB}, or saline were also used. Cells were treated for 24 hours with GFAP_{AB}/Drug/PEG-HCC and/or Il-13R_{AB}/Halo/PEG-HCC. Next, the cells were incubated for 1 hour with Rh123, BCECF-AM and Hoe, as previously described. Cells were then imaged at ×30 magnification using filters for Rh123, BCECF and Hoe.

FIG. 2A shows that there is a synergistic effect in dye accumulation using the two chemotherapeutic drugs and either of the xenobiotic pump inhibitors. When cells are incubated with Il-13R_{AB}/Inhibitor/PEG-HCC, there is an increase in dye retention. Furthermore, it can be seen that the pattern of dye retention is different in both cases. Likewise, both Doc and Vin differentially increase dye retention through competition with the three dyes for the xenobiotic pump transporters. In drug and inhibitor combinations, it can be noted that the greatest level of dye, especially BCECF, is seen in the presence of Doc and Halo.

FIG. 2B shows that the levels of living cells falls between more than about 50% and less than about 70% when the cells are treated with Vin or Doc in the presence of Halo, as compared with just Vin or Doc. Furthermore, it is shown in FIG. 2C that the dead cell numbers are elevated when Halo is used in conjunction with both Vin and Doc.

FIGS. 2D-2E show additional data relating to Halo-mediated potentiation of Vin, Doc and SN38 toxicity in HADES compositions. The data indicate that SN-38 toxicity is minimally affected by Halo. Without being bound by theory, such results suggest that this compound is mostly transported by a pump other than P-gp, as outlined in Table 1.

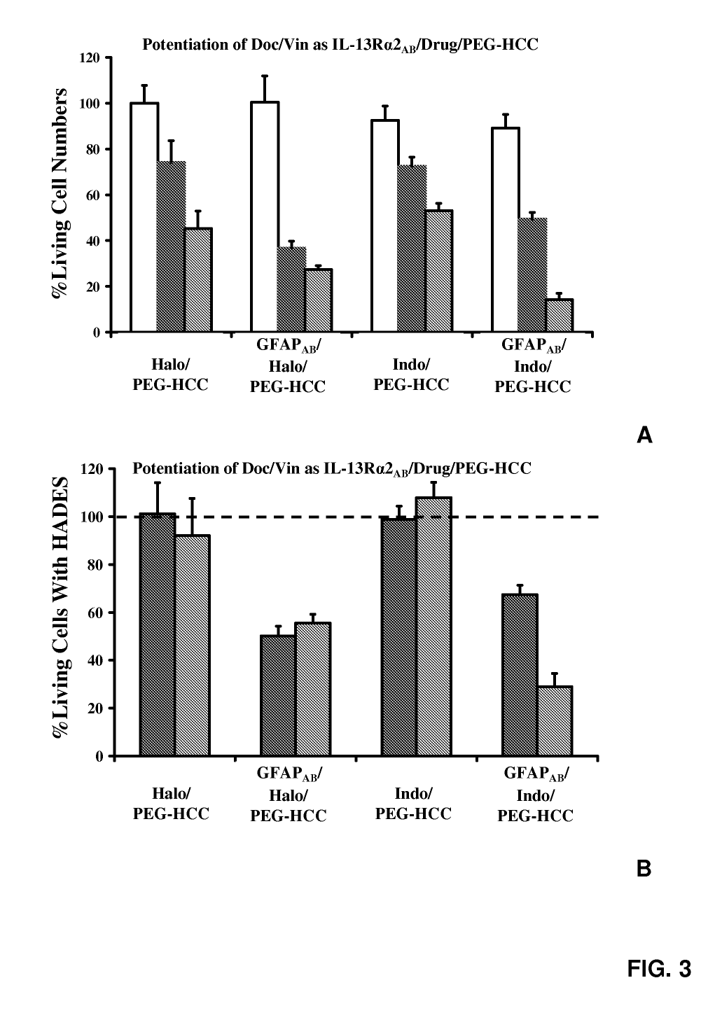

To confirm the potentiation effect of chemotherapeutic agents and pump inhibitors, Applicants performed a similar experiment to that shown in FIG. 2. In these experiments, Applicants examined the effects of both inhibitors on both chemotherapeutics. Applicants also inverted the targeting antibodies by utilizing GFAP_{AB}/(Indo or Halo)/PEG-HCC and Il-13R_{AB}/(Doc or Vin)/PEG-HCC. 2 μM Halo/PEG-HCC and Indo/PEG-HCC without antibody targeting were used as controls, which were added with and without 100 nM Vin or Doc as Il-13R_{AB}/Drug/PEG-HCC. The results are summarized in FIG. 3.

FIG. 3A shows that growing GBM cells for 24 hours in Indo/PEG-HCC (in the presence or absence of antibody targeting) causes a small drop in cell numbers that was statistically insignificant from growth in the presence of Halo/PEG-HCC (in the presence or absence of antibody targeting). 200 nM Il-13R_{AB}/Doc/PEG-HCC caused a drop in cell number to 75% and to 72% (the level seen in the controls) in the presence of (untargeted) Halo/PEG-HCC and Indo/PEG-HCC, respectively. However, targeting of Halo caused a large change in Doc toxicity. The inclusion of an antibody with Halo/PEG-HCC changed the toxicity from 75% to 37%. The inclusion of an antibody with Indo/PEG-HCC changed the Doc toxicity from 72% to 50%.

Vin toxicity was also potentiated in the presence of targeted xenobiotic pump inhibitors. Furthermore, the targeting of Halo dropped the cell numbers from 45% (control number, untargeted) to 27% (targeted). The combination of Vin and Indo proved to have the greatest toxicity, with Vin in the presence of untargeted Indo dropping cell numbers to 53% of the control value. However, when Indo was targeted to the GBM cells, this dropped to only 14%. In FIG. 3B, the same data is displayed in slightly modified form. Only the potentiating effect is shown, comparing targeted and untargeted xenobiotic pump inhibitors, so that the cell numbers in the presence of untargeted pump inhibitor are averaged to 100%. This shows that pump inhibition by Halo increases the toxicity of both Doc and Hal by approximately 50%, whereas Indo preferentially increases Vin toxicity by 70%, as compared to only 40% for Doc.

Drug Pump Inhibition Potentiates Chemotherapeutic Drug Action in Breast and Cervical Cancer Cells

Applicants also investigated whether the potentiation of Vin and Doc toxicity (by Halo and Indo) was applicable to other cancer types. Applicants obtained human breast and cervical cancer cells from the ATCC and grew them in 96 well format. In preliminary experiments, Applicants found that both cell types were more insensitive to both Doc and Vin than human GBM cells. Thus, Applicants used higher levels of both Doc and Vin in demonstration experiments.

In a pair of 96-well plates, confluent cancer cells were treated with 1 μM (Indo or Halo)/PEG-HCC and/or 1 μM (Doc or Vin)/PEG-HCC. The inhibitors were guided to the cell surface using anti-HER2 antibodies. The chemotherapeutics were guided using EGFR antibodies. After incubation for 24 hours, one plate was used to measure total cell protein, and the other was used in dye labeling studies (as described above). This was done for both HER2-neu+ breast and HER2-neu+ cervical cancer cells (where HER2-neu is human epidermal growth factor receptor-2).

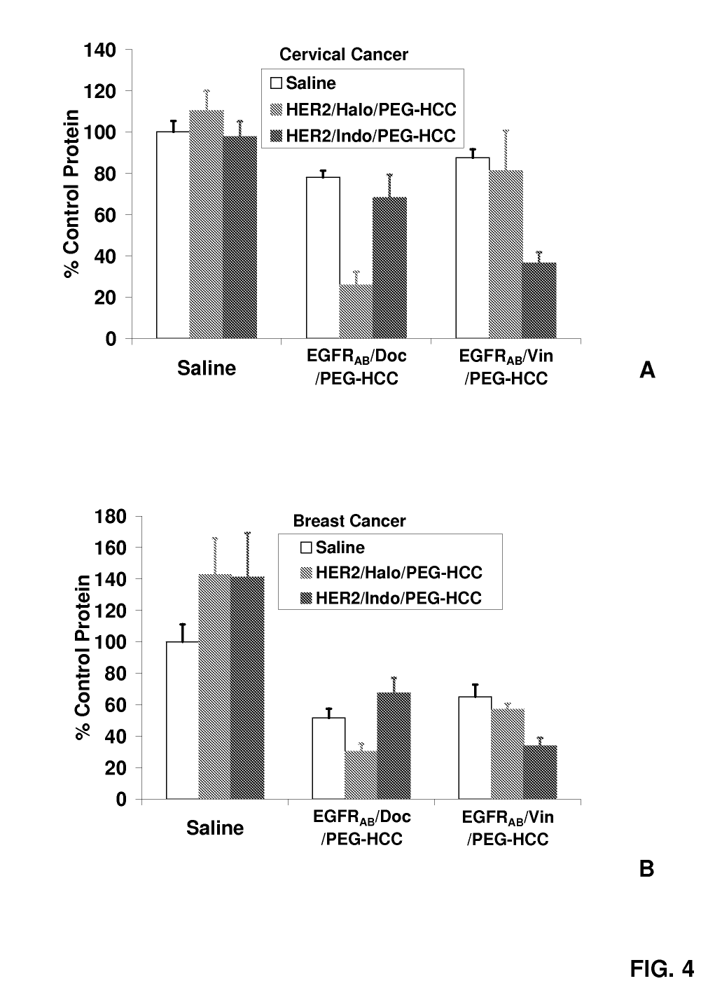

In FIG. 4, Applicants show that Halo and Indo potentiate the actions of both Vin and Doc in both cell types. In both cases, the Halo/Doc and Indo/Vin combinations show the greatest potentiation effect. FIG. 4A shows that 1 μM (Indo or Halo)/PEG-HCC has no effect on total cell protein (a measure of living cells). 100 nM Doc delivered using EGFR IgG yielded 78% of the control, whereas 100 nM Vin yielded 87% of the control. Halo increased the toxicity of Doc, lowering the total protein to 25% of the control. However, Halo only had a slight effect on Vin toxicity. Indo increased the toxicity of Doc slightly (from 78% to 68%) and Vin greatly (from 87% to 36%). In the panels of FIG. 4C, the dye retention of these cervical cancer cells is shown under the same conditions. All four compounds cause a significant brightening of the cells. Furthermore, it is clear that Doc/Halo, Vin/Halo and Vin/Indo show significant increases in RBG dye retention.

FIG. 4B shows that 1 μM (Indo or Halo)/PEG-HCC causes an unexpected 25% increase in total cell protein. 100 nM Doc delivered using EGFR IgG yielded 51% of the control, whereas 100 nM Vin yielded 65% of the control. Halo increased the toxicity of Doc, thereby lowering the total protein to 30% of the control. However, Halo had no potentiating effect on Vin toxicity. Indo slightly decreased the toxicity of Doc (from 51% to 68%) and greatly increased the toxicity of Vin (from 65% to 33%). In the panels of FIG. 4D, the dye retention of these breast cancer cells is shown under the same conditions. All four compounds cause a significant brightening of the cells. The inhibitor and chemotherapeutic combination panels also indicate that dye retention is potentiated in all four combinations/permutations.

In sum, Example 1 demonstrates that chemotherapeutic drug and xenobiotic drug pump inhibitor pairs can be selected to increase the toxicity of cancer treatment. Moreover, the changes in dye retention are indicative of a mechanism of chemotherapeutic drug resistance based on the function of xenobiotic drug pumps in different cancer types.

### Example 2

### Formulation and Use of Therapeutic Compositions for Treating Cancer

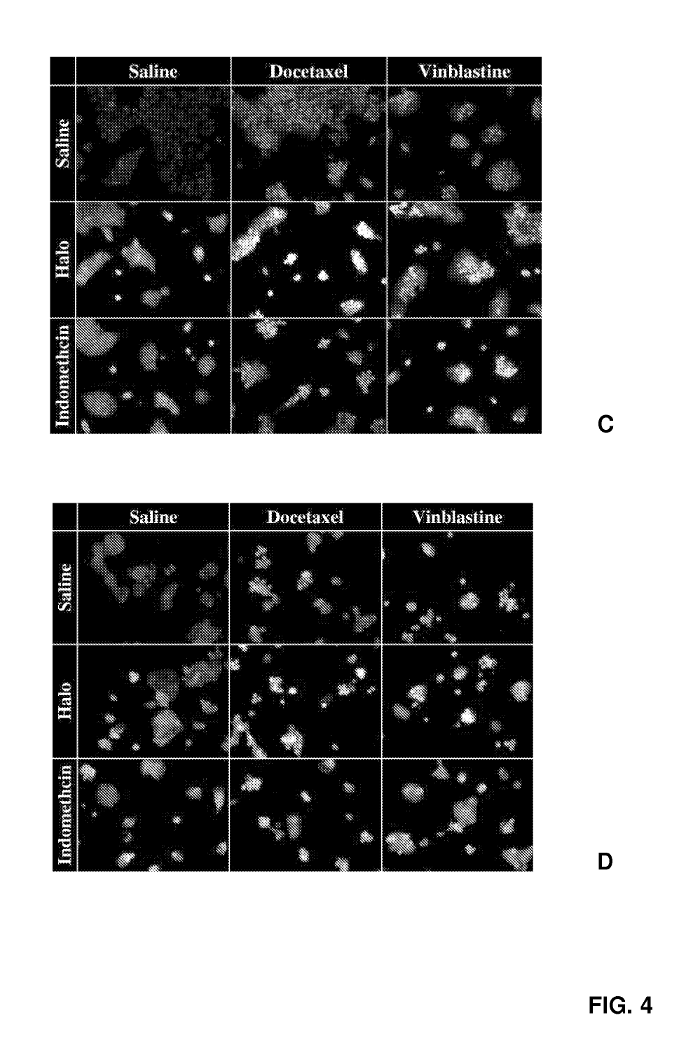

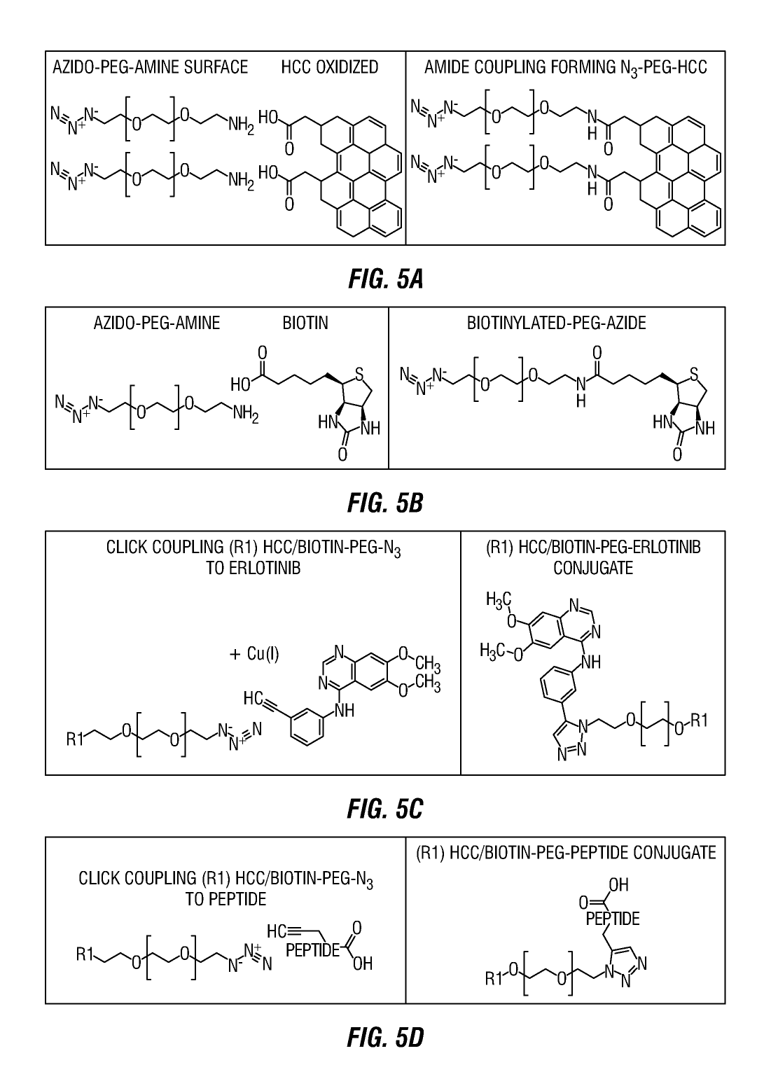

This example illustrates the formulation of PEG-HCC constructs for delivery of therapeutic compositions. In particular, this Example pertains to the formulation of bi-functional, cell-type specific, targeting reporters or vector-docking linkers. FIG. 5 shows how it is possible to rapidly and efficiently synthesize a reporter probe that has the same cell surface binding properties as a targeted nanovector.

The free ends of PEGs or PEG-HCCs can be furnished with a wide range of functional groups, thereby allowing covalent attachment of targeting moieties or moieties that have other functions. See, e.g., Chemical Society Reviews 41(2012):2971-3010. For instance, the moieties can facilitate the passage of a PEG-HCC nanovector through an intact blood brain barrier by the addition of Adamante as a moiety.

Likewise, PEGs can be used to create a common linker chain so that one end can be endowed with specificity toward a specific cell surface protein, and the other end can be appended to one or more of the following molecules: 1) a reporter, for the use of quantification of specific membrane protein in a tissue section/cell culture/protein homogenate; and/or 2) an HCC drug carrying nanovector. The reporter function has utility in that it allows pre-screening of a cancer biopsy sample for the ability of different peptides or docking molecules to bind to the surface. This would allow a physician to screen and quantify the levels of different surface proteins in a particular patient, or a particular tumor. The physician could then make an informed decision as to the best drug targeting strategy.

In this example, biotin is the reporter and HCC is the nanovector. Applicants can show how the reporter/nanovector are coupled to either targeting peptides or known compounds that bind surface proteins known to be over expressed in cancer cells.

Linkers

In this Example, the linker is azido-PEG-amine (N₃-PEG-NH₂). HCCs are oxidized carbon nanotubes and their two dimensional graphene structure is pockmarked with carboxylate groups that are covalently attached to Poly(ethylene glycol) bis(amine) (NH₂-PEG-NH₂) via the formation of an amide, typically using carbodiimide coupling. However, many other methodologies are available (Tetrahedron 60 (2004) 2447-2467). Applicants have prepared and used this same azido-PEG-amine (N₃-PEG-NH₂) linker previously (Nano 4 (2010) 4621-4636).

Connection of Linker to HCC

In FIG. 5A, Applicants show how N₃-PEG-NH₂ is connected to HCC to generate N₃-PEG-HCC. Thus, a nanovector is connected to an azido group at the end of a fexible linker.

Connection of Linker to a Reporter

In the same manner as the nanovector is connected to N₃-PEG-NH₂, one can also couple a biotin reporter to a PEG linker that bares the same common structure as N₃-PEG-HCC. The exemplary scheme is illustrated in FIG. 5B.

Attaching Docking Moieties to HCCs

As an example of the covalent attachment of targeting moieties to HCCs, Applicants have utilized a classic “click” reaction. This click reaction comprises the copper-catalyzed azide-alkyne cycloaddition to form a 1,4-disubstituted-1,2,3-triazole linkage (Angewandte Chemie International Edition 48 (2009) 9879-9883).

Targeting Peptides

Targeting peptides that have the ability to bind to specific surface expressed proteins can be synthesized so that they include an N, X or C terminal ethyne (—C≡CH). The peptides can be attached using the click reaction to form a stable, triazole linkage. See FIG. 5C.

Targeting Substrate/Inhibitor Compounds

Drug compounds that bind to over-expressed cell membrane proteins and incorporate an ethyne group (—C≡CH), or which can be modified to include such a moiety, can be attached using the same click chemistry. Herein, Applicants use the example of Erlotinib, which is an Epidermal Growth Factor Receptor (EGFR) inhibitor that binds to the WT receptor (and common mutants) with a K_{D} of less than 12 nM. Applicants also utilize the commonly found truncated form of this protein, EGFRv_{III}. See FIG. 5D.

Additional Targeting

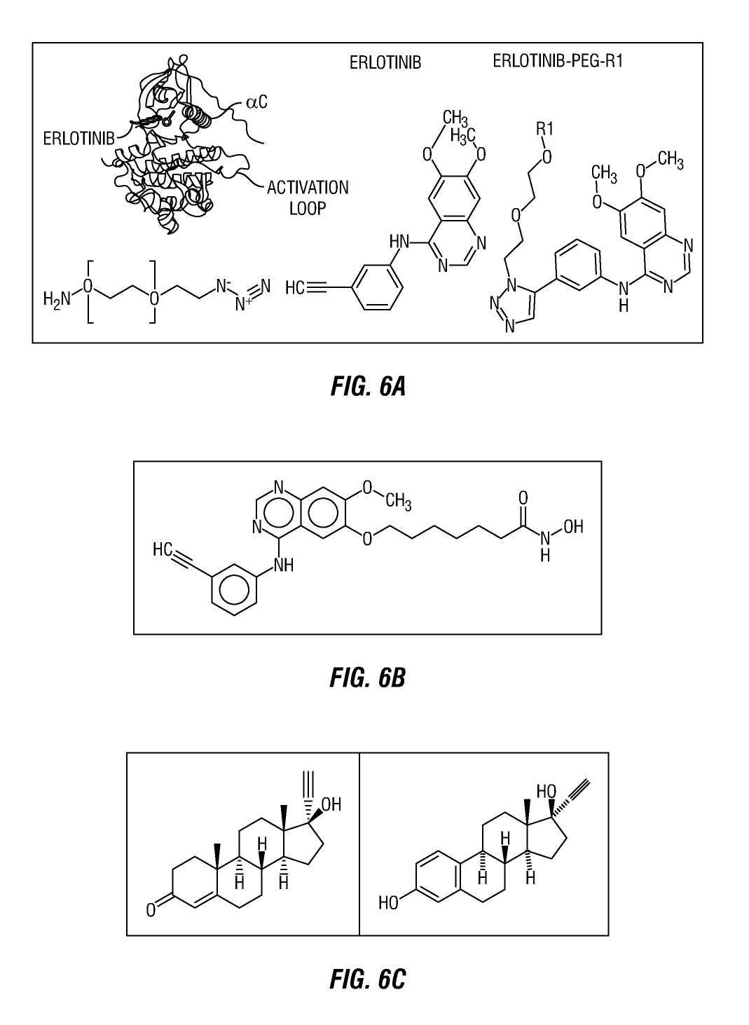

Applicants have also made HADES compositions that can target various EGFRs on cancer cells. For instance, FIG. 6A shows the coupling of EGFR antagonist Erlotinib to Azido-PEG-HCC/Biotin via click chemistry. FIG. 6A also shows the native structure of Erlotinib, which contains an ethyne group that is known to project into the outer bulk phase in the X-Ray crystal structure of the antagonists/EGFR complex (Protein Data Bank (http://www.rcsb.org/pdb/) entry 1M17).

Likewise, FIG. 6B shows the structure of CUDC-101 containing click chemistry available ethyne groups that can be used to generate potent multi-targeted HADES compositions. FIG. 6C shows how a membrane androgen receptor can be ligated with Ethisterone (left panel) to treat therapy-resistant prostate cancer, and Ethinylestradiol (right panel) to treat breast cancer or colorectal carcinoma.

These examples demonstrate that the HADES compositions of the present disclosure can be used to target different cancer cell surface receptors. The compositions can also be used to target and visualize cell surface antigens at the same time.

### Example 3

### Using Peptides to Target Cancer Cell Surface Receptors

Applicants have previously demonstrated that antibodies can be used to target PEG-HCC to cell surfaces. In particular, Applicants demonstrated that about 1-2 antibodies can get appended to each PEG-HCC. See, e.g., ACS Nano 6 (2012) 3114-3120.

However, the use of antibodies for targeting purposes has numerous limitations. For instance, once cannot inject mouse antibodies into a patient due to adverse immunological responses. Furthermore, humanized mouse monoclonal antibodies each cost $250,000 to establish. In addition, such humanized antibodies may have different specificities and affinities.

Applicants have determined that a viable alternative to the use of antibodies to target PEG-HCC to cell surfaces is the use of peptides. Phage display libraries make use of assisted evolutionary selection pressure to generate peptidyl sequences that bind to a particular epitope of interest. There are a large number of peptides that have been identified as binding with high affinity and specificity for particular tyrosine kinase receptors, such as Epidermal Growth Factor Receptor (EGFR), EGFRv_{III}, Neuropilin-1, Interleukin-4 Receptor α, Vascular Endothelial Growth Factor Receptor, Integrins αvβ3 and α5β, Gastrin-releasing peptide receptor, c-Met and Prostate Specific Membrane Antigen. Since many specific tyrosine kinase receptors are either up-regulated or only present on cancer cells (i.e. EGFRv_{III}), tyrosine kinase receptor binding peptides could also be used for targeting these cancer cells.

As set forth in more detail herein, Applicants have shown that they can covalently couple peptide/drug antagonists to PEG-HCC nanovectors for effective and specific delivery of active agents and enhancers to desired cancer cells.

Peptides that have been demonstrated to bind to tyrosine kinase receptor complexes have been modified with an N-terminal ethyne moiety and attached to azido modified PEG using ‘Click’ coupling chemistry. This allows Applicants to make two types of constructs, peptidyl-PEG-HCC and peptidyl-PEG-Biotin. The method also avoids the use of antibodies and problems with immunogenicity.

In particular, Applicants have shown that they can use artificial and natural peptides to bind to surface receptors that are up-regulated in cancer cells. In fact, many receptors that are up-regulated on the surface of cancer cells bind to specific peptides with high affinity. For instance, Table 2 shows a number of cell surface receptors that are known to be highly expressed on cancer cell surfaces, and the specific peptide sequence(s) that bind to these receptors with high affinity.

TABLE 2
Examples of potential cancer cell surface receptors and
peptide sequence(s) that bind to the receptors with
high specificity and affinity.

| Cell Surface receptor | Peptide |
|---|---|
| Epidermal Growth Factor | YHWYGYTPQNVI |
| Receptor; EGFR | YRWYGYTPQNVI |
| EGFRv_{III} | FALGEA |
| | FALGEA |
| Neuropilin-1 | NYQWVPYQGRVPYPRGGGKL |
| | ATWLPPR |
| Transferrin receptor | THRPPMWSPVWPGGG |
| Interleukin-4 Receptor | KQLIRFLKRLDRNGGG |
| alpha | |
| Prostate Specific | WQPDTAHHWATLK |
| Membrane Antigen | WQPDTAHHWATLKKLTAWHHATDPQW |
| Vascular Endothelial | CGYWLTIWGC |
| Growth Factor R | |
| VEGF-2 & VEGF-3 | |
| Human Epidermal | YCDGFYACYMDA |
| Growth Factor | |
| Receptor 2 | |
| Integrin αᵥβ3 | DFKLFAVYIKYR |
| and α5β1 | DFKLFAVTIKYR |
| Gastrin-releasing | QWAVGHLM |
| peptide receptor | QWAVGHL-Ethyl |
| c-Met | YLFSVHWPPLKA |

Furthermore, the peptides can be synthesized by the use of “click chemistry”, as illustrated in Table 3 and FIG. 5D. For instance, ethyne groups containing N-terminus and C-terminus moieties can be used to propagate peptide synthesis or coupling reactions. In some embodiments, the ethyne groups may be coupled to another molecule by conventional azide coupling.

TABLE 3
Use of ethyne groups for peptide synthesis and coupling via “click chemistry.”

| N-Terminus, γ-Lys | C-Terminus, γ-Asp or γ-Glu |
|---|---|
| 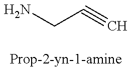 | 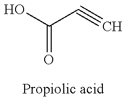 |

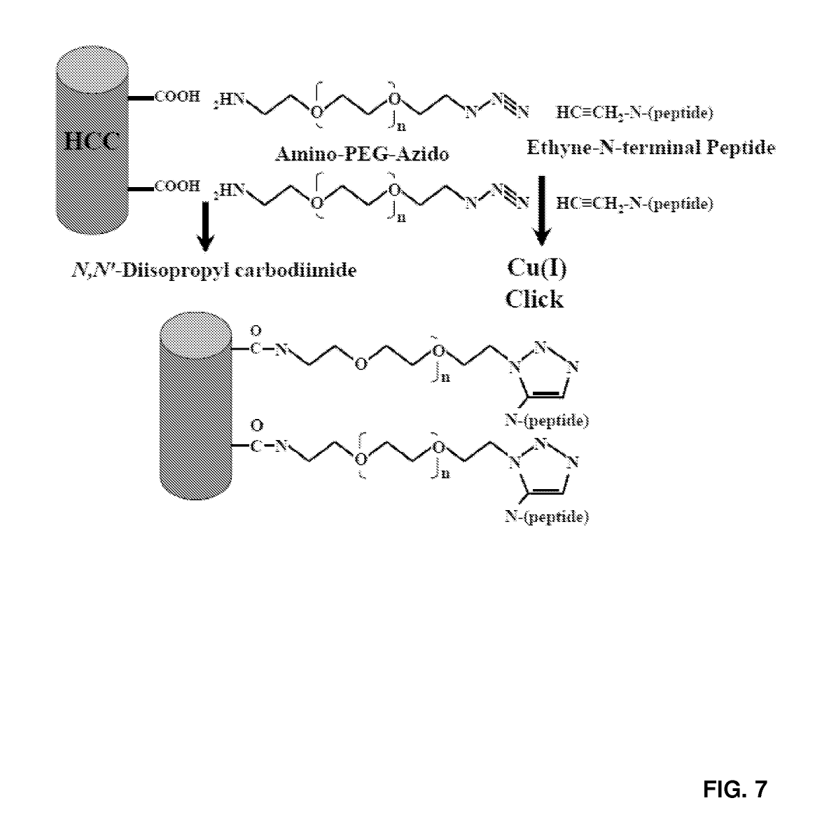

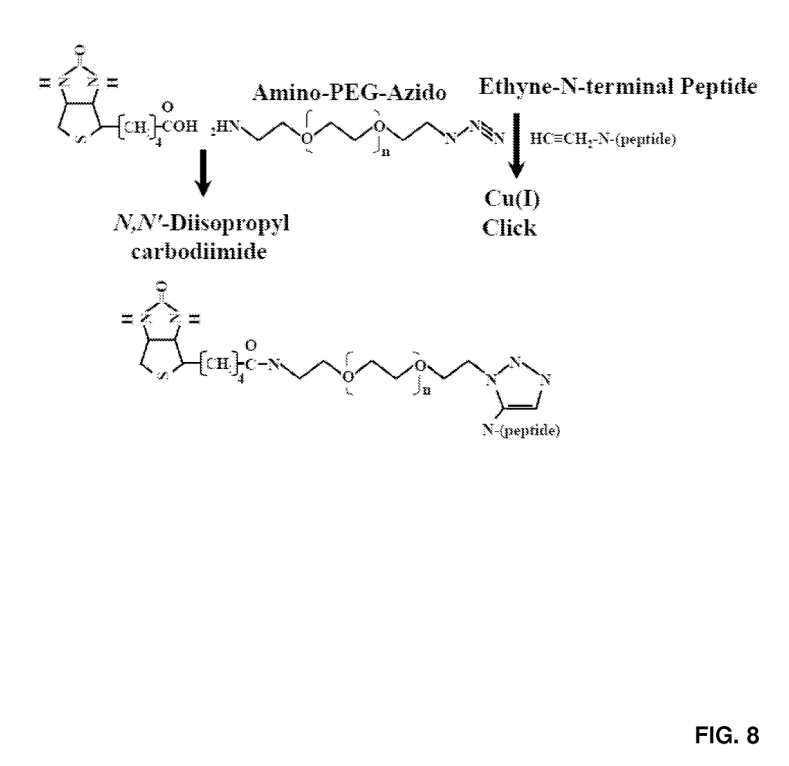

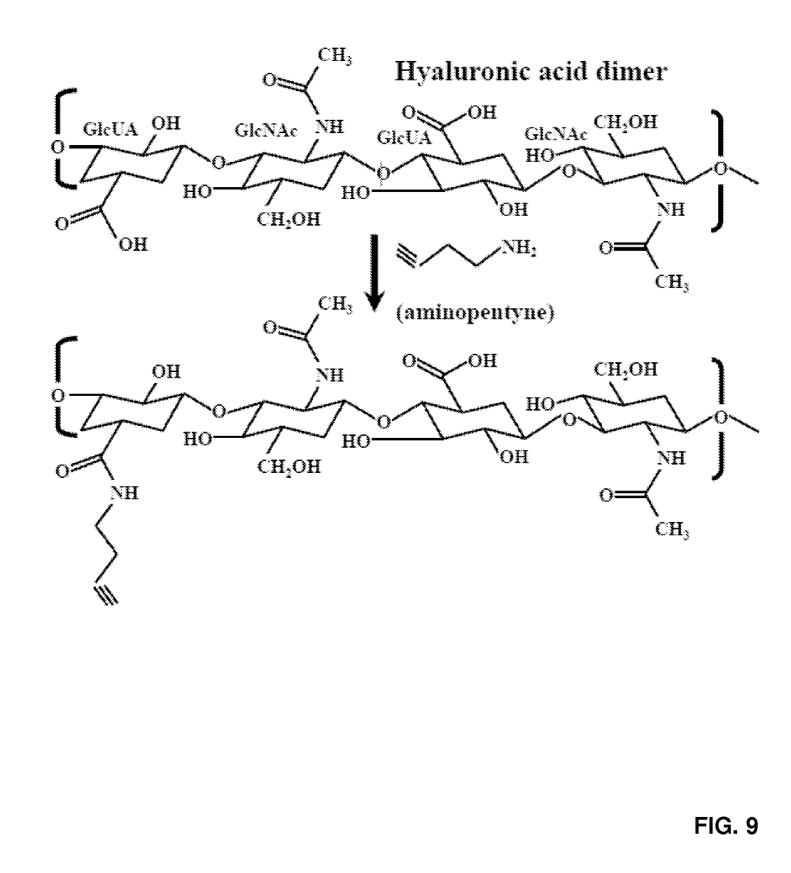

Additional reaction schemes for synthesizing peptidyl PEG-HCC and peptidyl PEG-Biotin by the use of “click chemistry” are illustrated in FIGS. 7-8. FIG. 9 shows how hyaluronic acid may be modified with aminopentyne so as to be able to be connected via “click chemistry” to azido-PEG-HCC and azido-PEG-Biotin.

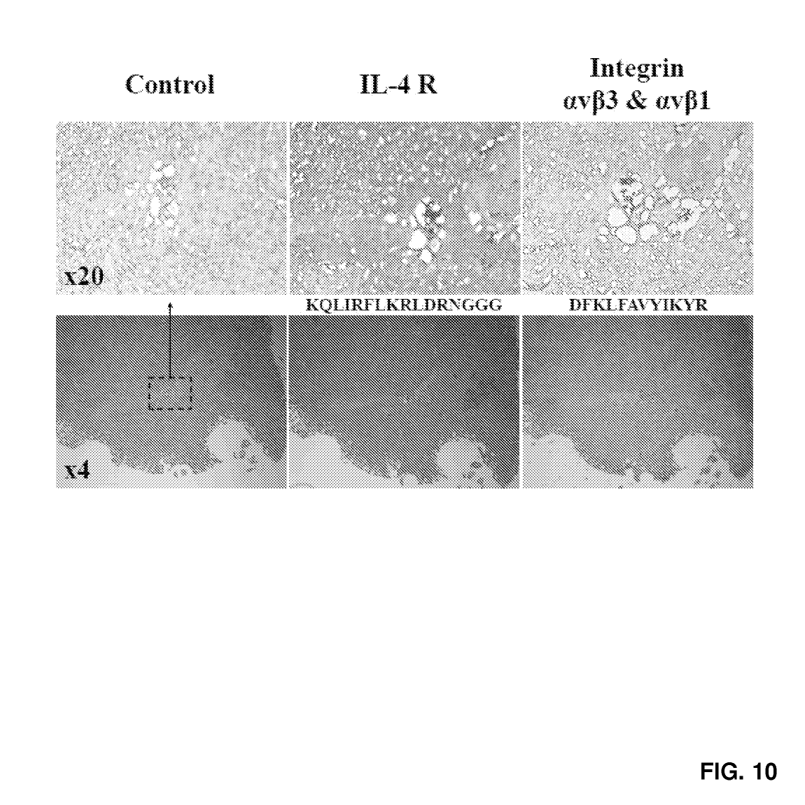

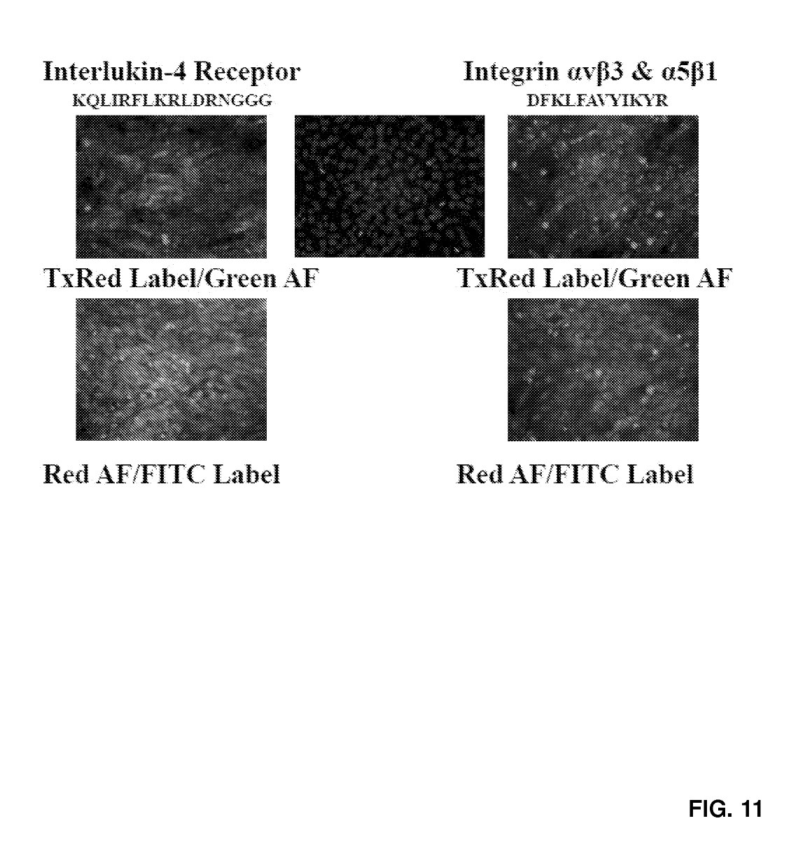

Furthermore, as illustrated in FIGS. 10-11, various peptides that are linked to HADES compositions can bind to cancer cell surface receptors. For instance, FIG. 10 provides images illustrating that biotin-PEG-peptide molecules bind to GBM cells (i.e., biopsy samples from BT111 cells). In this experiment, the GBM cells were fixed, waxed, and sliced. Thereafter, the GBM cells were placed on slides, dewaxed, and rehydrated. The nuclei of the GBM cells were labeled with haematoxylin without utilizing detergents. Thereafter, the cells were blotted with Avidin/Biotin and incubated with biotinylated-PEG-Peptides. After washing the label with Streptavidin-HRP, the cells were visualized using a DAB (diaminobenzidine) kit from Dako (Dako. Carpinteria, Calif. USA). Likewise, FIG. 11 provides additional images illustrating that biotin-PEG-peptide molecules bind to the surfaces of GBM cells (i.e., biopsy samples from BT111 cells). In this experiment, GBM cell cultures were treated with Hoechst, which labels nuclear DNA blue, and fixed in PFA without utilizing detergents. The cells were then incubated for 30 minutes with biotinylated-PEG-Peptide. Half of the cells were labeled with FITC-Avidin, which labels the biotin-marker green. The other half of the cells were labeled with Texas Red-Avidin, which labels the biotin-marker red. The cells were visualized with Red/Green/Blue light. The results indicate that either red or green (Tex Red or FITC) is orders of magnitude greater than non-specific avidin binding/background fluorescence.

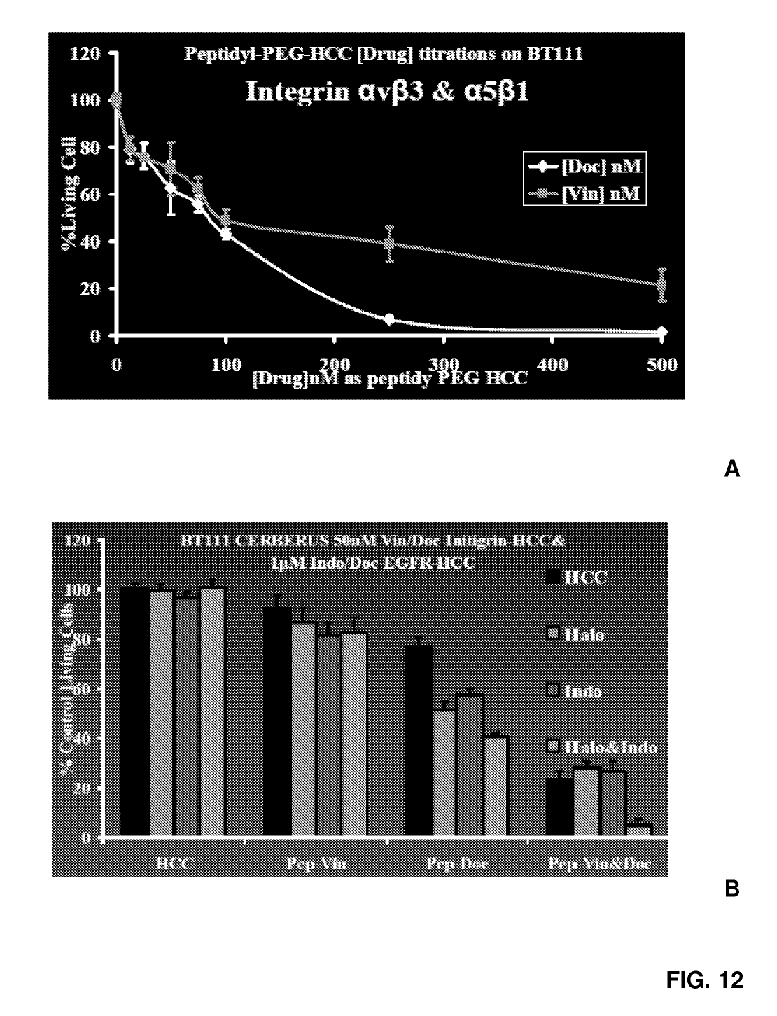

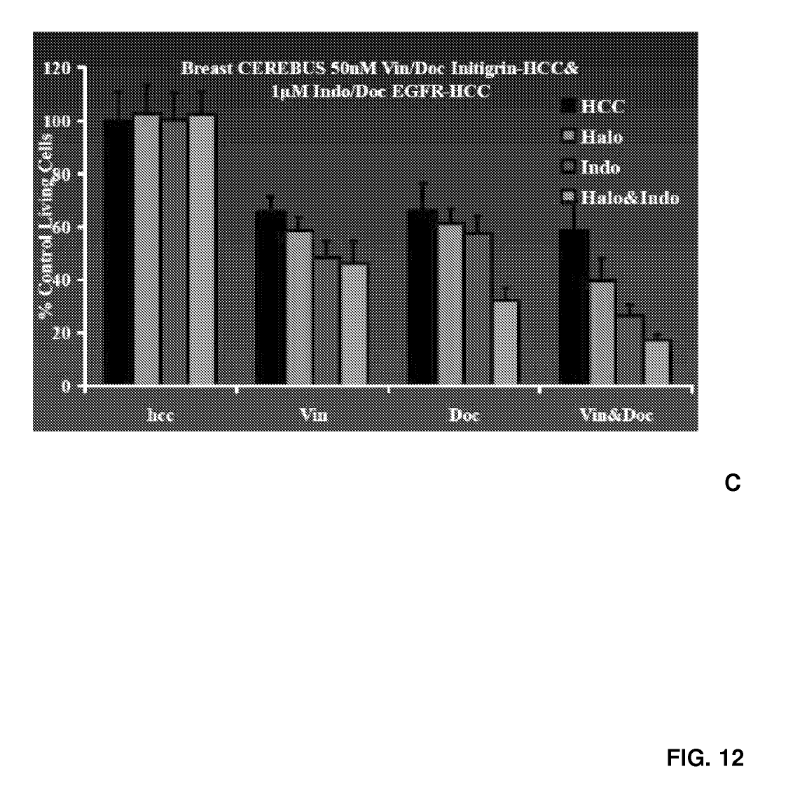

More importantly, Applicants have demonstrated that peptide-linked PEG-HCCs can be utilized as effective HADES compositions to target cancer cells. For instance, FIG. 12A provides a chart indicating that peptidyl-PEG-HCCs loaded with Vin or Doc can target GBM cells (i.e., BT111 cells). In all cases, living cell numbers were calculated at n=6 individual wells and assayed using Hoe. Cells were incubated for 24 hours with Doc or Vin and targeted to the cell surface with peptidyl-PEG-HCC. The targeting peptidyl sequence was DFKLFAVTIKYR, which targets Intigrins αvβ3 and α5β. FIGS. 12B-12C provide data illustrating that drug pump inhibitors Halo and Indo potentiate the effects of Vin and Doc on GBM and breast cancer cells. Cells were incubated with PEG-HCC (control), 50 nM Doc or Vin, or Doc and Vin as peptidyl-PEG-HCC (using DFKLFAVTIKYR targeting Intigrins αvβ3 and α5β). Additionally, these four incubants were treated with PEG-HCC (control), 1 μM Halo or Indo, or Halo and Indo as peptidyl-PEG-HCC (using YRWYGYTPQNVI targeting EGFR).

These low levels of chemotherapeutic compounds only caused a limited level of cell death at 24 hours in gliomal cells, but were individually more toxic in breast cancer cells (black bars). In gliomal cells, Vin toxicity was moderately enhanced by co-incubation with Halo (red bars) and more than doubled in the presence of Indo (blue bars). The presence of both pump inhibitors (pink bars) did not lead to a greater level of toxicity than Indo alone. In gliomal cells, Doc toxicity was more enhanced by co-incubation with Halo (red bars) than with Indo (blue bars), but both pump inhibitors led to a greater level of toxicity than Halo or Indo alone. Finally, co-incubation of Doc and Vin together was more toxic than the individual compounds in glioma. This toxicity was greately enhanced by the presence of Halo and Indo, but not by these drug pump inhibitors singly.

A different pattern of potentiation of Doc and Vin is found in human breast cancer cells, than was observed in glioma. Vin and Doc added together were only slightly more toxic than the individual chemotherapeutic drugs. Vin was potentiated by Indo and Doc by both Indo and Halo, but greatly by both on combination. Breast cancer cells were very sensitive to a combination of both drugs and both drug pump inhibitors.

Synthesis of Azidopolyethylene Glycol Amine

A thick-walled reaction tube was oven-dried, fitted with a stir bar and septum, and pump/filled with nitrogen three times. 10 mL of freshly distilled THF and 4.44 mL of potassium bis(trimethylsilyl)amide (2.22 mmol) were added. Next, the solution was cooled to −78° C. using a dry ice/acetone bath. Separately, a 25 mL graduated cylinder was filled with 200 mg CaH₂, fitted with a septum, and cooled to −78° C. Ethylene oxide (5 mL, 100 mmol) was condensed in the cylinder and transferred to the reaction tube via cannula. The septum on the reaction tube was removed and the tube was quickly sealed. The reaction was stirred at 60° C. for 16 h, during which time the reaction mixture gradually turned a rusty orange-brown and became visibly viscous. The reaction was then cooled to room temperature. N,N-diisopropylethylamine (1.2 mL, 7 mmol) followed by p-toluenesulfonyl chloride (1.27 g, 6.67 mmol) were then added to the reaction in single portions. The light brown reaction mixture was stirred at 60° C. for 16 h. The mixture was then poured into a solution of sodium azide in H₂O to give a biphasic mixture. The mixture was heated at 90° C. for 4 h and then extracted with diethyl ether (3×40 mL) and chloroform (4×40 mL). The chloroform extracts were combined, dried under magnesium sulfate, evaporated under reduced pressure to 30 mL, and treated with diethyl ether (150 mL). The product crystallized as white needles upon cooling at −20° C. The solid was collected on a PTFE membrane, washed with diethyl ether and dried in vacuo to give 3.7 g of azidopolyethylene glycol amine. GPC analysis gave a molecular weight of 5864.

Synthesis of Biotinylated Polyethylene Glycol

Biotin (9.4 mg, 0.038 mmol) and N,N′-dicyclohexylcarbodiimide (7.9 mg, 0.038 mmol) were dissolved in dry N,N′-dimethylformamide. The resulting solution was stirred at room temperature for 30 min. 4-dimethylaminopyridine (2 flakes) was then added to the solution. This was followed by the addition of azidopolyethylene glycol amine (0.150 g, 0.026 mmol). Next, the reaction mixture was stirred for 16 h at room temperature. The reaction mixture was then transferred to dialysis tubing (1000 MWCO) and dialyzed in continuously flowing D.I. water for 5 days. The water was filtered and evaporated under reduced pressure. The residue was dissolved in 2 mL of chloroform and precipitated with cold diethyl ether to produce 0.120 g of biotinylated polyethylene glycol.

General Synthesis of Peptide-Functionalized Biotinylated Polyethylene Glycol

Alkynyl-functionalized peptide (e.g., HC≡C—CO—YHWYGYTPQNVI, 0.2 mg, 0.13 μmol) was dissolved in 0.2 mL of a 1:1 mixture of tert-butanol and D.I. water. Biotinylated polyethylene glycol (10 mg) was dissolved in 6 ml of a 1:1 mixture of tert-butanol and D.I. water. Copper sulfate (52 μL of a 2.5 mM solution in water) and sodium ascorbate (52 μL of a 2.5 mM solution in water) were then added. The reaction was stirred for 2 days at room temperature. Next, the mixture was dialyzed in continuously flowing D.I. water for 2 days.

Synthesis of Azide-Functionalized PEG-HCCs

HCCs (30 mg, 2.5 mmol of carbon) were dissolved in N,N′-dimethylformamide with the aid of a bath sonicator for 30 min. N,N′-dicyclohexylcarbodiimide (205 mg, 1 mmol), methoxypolyethlyene glycol amine (125 mg, 0.025 mmol), azidopolyethylene glycol amine (147 mg, 0.025 mmol) and 4-dimethylaminopyridine (2 flakes) were then added. The reaction was stirred for 24 h. The solution was purified by dialysis in N,N′-dimethylformamide for 2 days. This was followed by dialysis in continuously refreshed D.I. water for 5 days. The resulting solution was then passed through a PD-10 column to yield a solution of azide-functionalized PEG-HCCs.

Synthesis of Peptide-Functionalized PEG-HCCs

Alkynyl-functionalized peptide (e.g., HC≡C—CO—YHWYGYTPQNVI, 0.8 mg, 0.52 μmol) was dissolved in 0.8 mL of a 1:1 mixture of tert-butanol. D.I. water was then added to a 3 mL solution of azide-functionalized PEG-HCCs. To this solution was added tert-butanol (3 mL), copper sulfate (208 μL of a 2.5 mM solution in water) and sodium ascorbate (208 μL of a 2.5 mM solution in water). The reaction was stirred for 2 days at room temperature. The mixture was dialyzed in continuously flowing D.I. water for 1 day. Excess copper was removed by treatment with sodium sulfide and calcium hydroxide.

Loading Peptide-Functionalized PEG-HCCs with Drugs (e.g. Docetaxel)

Docetaxel (0.2 mL of a 1 mg/mL solution in methanol) was added dropwise into a rapidly stirring solution of peptide-PEG-HCCs in deionized water (2 mL, 331 mg/L concentration of core HCC). The mixture was stirred at room temperature for 16 h. To remove the methanol, the solution was concentrated under reduced pressure to 1 mL and reconstituted to 2 mL with D.I. water to give a final docetaxel concentration of 0.1 mg/mL.

### Example 4

### Utilization of HADES Compositions for In Vivo Breast Cancer Treatment

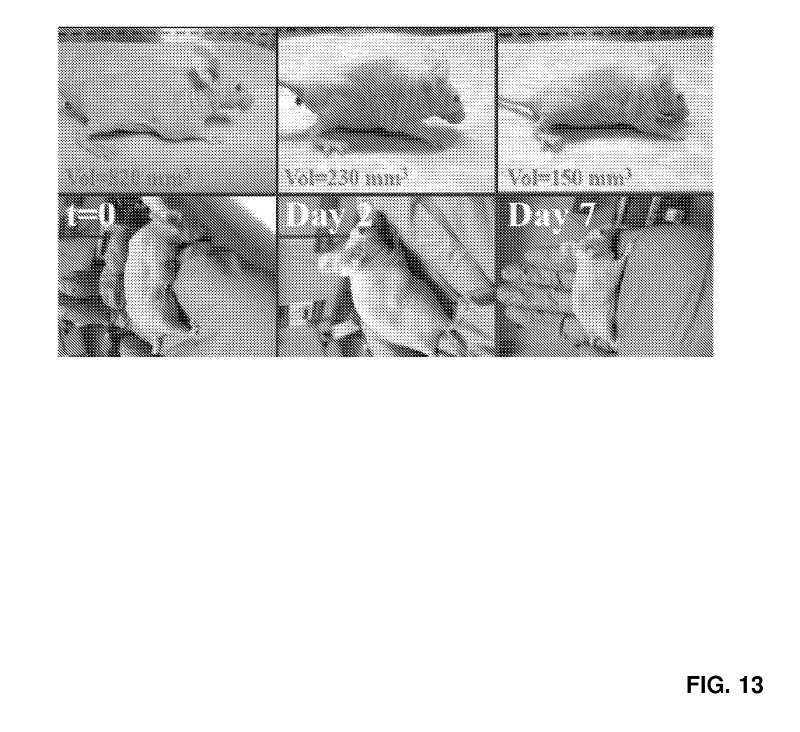

In this Example, Applicants have demonstrated that the HADES compositions of the present disclosure can be used to treat breast cancer in a nude mouse model of human breast cancer. For instance, FIG. 13 provides images indicating that HADES compositions containing Vin, Doc, Halo and Indo can be used to treat breast cancer in a nude mouse model of human breast cancer

500,000 breast cancer cells (ZR-75) were first suspended in Matrigel™. The cells were then injected into a nude mouse flank. After 25 days, a tumor of 820 mm³ had grown. Integrin targeting peptidyl-PEG-HCC (DFKLFAVTIKYR) was loaded with chemotherapeutic drugs Vin and Doc. EGFR targeting peptidyl-PEG-HCC (YRWYGYTPQNVI) was loaded with pump inhibitors Halo and Indo. The mouse had a single tail vain injection that consisted of 200 nM Doc and Vin, and 900 nM Halo and Indo. The tumor volume demonstrated tumor shrinkage of more than 80% after 7 days of treatment.

### Example 5

### Treatment Mechanisms

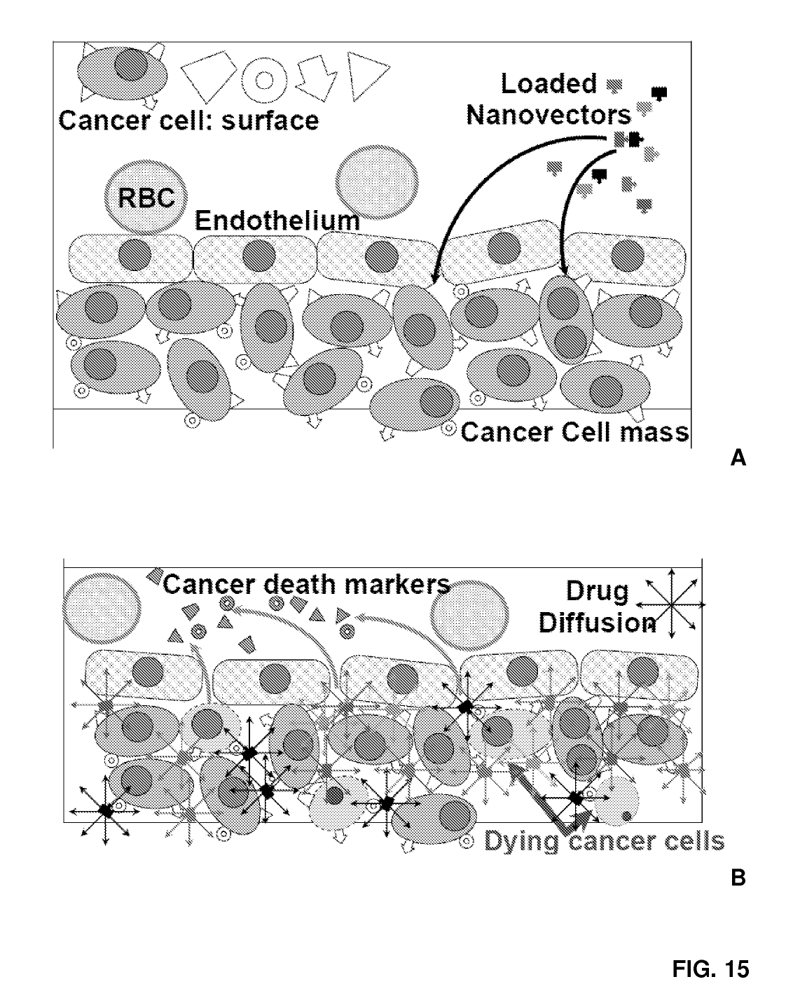

In FIG. 15, Applicants demonstrate how treatment could work in an individual manner. In this Example, a patient is treated with four different targeting nanovectors. The choice of targeting comes from screening a biopsy, or from an examination of primary cultures derived from the patient's tumor, or even from in vivo imaging when the biotinylated-linker-probe is linked to an MRI/PET detectable visualization group. The personalized choice of targeting could include humanized IgG, peptides, or small receptor antagonists attached to the PEG-HCC. These are loaded with a chemotherapeutic drug and a pump inhibitor and delivered into the vasculature, possibly by direct site injection. Thereafter, the drug/pump inhibitor contents of the nanovectors are released at the surface of the cancer cell plasma membrane. Next, the contents diffuse into the cells binding the constructs. Some of the contents may also diffuse to nearby cells.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A therapeutic composition for targeting tumor cells, wherein the therapeutic composition comprises:
a plurality of nanovectors;
one or more active agents associated with the nanovectors,
wherein the one or more active agents have activity against the tumor cells;
one or more active agent enhancers associated with the nanovectors; and
one or more targeting agents associated with the nanovectors,
wherein the one or more targeting agents have recognition activity for one or more markers of the tumor cells.

2. The therapeutic composition of claim 1, wherein the one or more active agents and the one or more active agent enhancers are associated with same nanovector molecules.

3. The therapeutic composition of claim 1, wherein the one or more active agents and the one or more active agent enhancers are associated with different nanovector molecules.

4. The therapeutic composition of claim 3,
wherein the one or more active agents are associated with a first nanovector molecule,
wherein the first nanovector molecule is associated with a first targeting agent, and
wherein the one or more active agent enhancers are associated with a second nanovector molecule,
wherein the second nanovector molecule is associated with a second targeting agent.

5. The therapeutic composition of claim 1, wherein the one or more active agents are non-covalently associated with the nanovectors.

6. The therapeutic composition of claim 1, wherein the one or more active agents are covalently associated with the nanovectors.

7. The therapeutic composition of claim 1, wherein the one or more targeting agents are non-covalently associated with the nanovectors.

8. The therapeutic composition of claim 1, wherein the one or more targeting agents are covalently associated with the nanovectors.

9. The therapeutic composition of claim 1, wherein the one or more active agent enhancers are non-covalently associated with the nanovectors.

10. The therapeutic composition of claim 1, wherein the one or more active agent enhancers are covalently associated with the nanovectors.

11. The therapeutic composition of claim 1, wherein the nanovectors comprise hydrophobic domains and hydrophilic domains,
wherein the one or more active agents and the one or more active agent enhancers are associated with the hydrophobic domains, and
wherein the one or more targeting agents are associated with the hydrophilic domains.

12. The therapeutic composition of claim 1, wherein the nanovectors are selected from the group consisting of single-walled carbon nanotubes, double-walled nanotubes, triple-walled nanotubes, multi-walled nanotubes, ultra-short nanotubes, graphene, graphene nanoribbons, graphite, graphite oxide nanoribbons, carbon black, oxidized carbon black, hydrophilic carbon clusters, graphene quantum dots, and combinations thereof.

13. The therapeutic composition of claim 1, wherein the nanovectors are functionalized with a plurality of solubilizing groups.

14. The therapeutic composition of claim 14, wherein the solubilizing groups are selected from the group consisting of polyethylene glycols, poly(p-phenylene oxide), polyethylene imines, poly(vinyl amines), and combinations thereof.

15. The therapeutic composition of claim 1, wherein the nanovectors comprise an ultra-short single-walled carbon nanotube, wherein the nanotube is functionalized with a plurality of solubilizing groups.

16. The therapeutic composition of claim 1, wherein the nanovectors comprise a polyethylene glycol functionalized hydrophilic carbon clusters (PEG-HCC).

17. The therapeutic composition of claim 1, wherein the one or more active agents are hydrophobic.

18. The therapeutic composition of claim 1, wherein the one or more active agents are selected from the group consisting of cis-platin, SN-38, vinblastine, daunorubicin, paclitaxel, docetaxel, doxorubicin, epirubicin, vincristine, iadarubicin, mitoxantrone, oxaliplatin, topotecan, etoposide, erlotinib, ethisterone, ethinylestradiol, 1,2,3,4-tetrahydronaphthalene-2,3-diamine, 2,2-dichloro-octahydrocyclohexa 1,3-diaza-2-platinacyclopentane, 2,2-dichloro-hexahydro-naphtho1,3-diaza-2-platinacyclopentane, 4,4-dichloro-3,5-diaza-4-platinatetracycloheptadecahexaene, nitrogen mustards, spermine mustards, estrogen mustards, cholesterol mustards, and combinations thereof.

19. The therapeutic composition of claim 1, wherein the one or more active agent enhancers comprise one or more drug transport pump inhibitors.

20. The therapeutic composition of claim 19, wherein the one or more active agent enhancers comprise xenobiotic drug pump inhibitors.

21. The therapeutic composition of claim 20, wherein the one or more active agent enhancers are selected from the group consisting of fumitremorgan C, indomethacin, 6-thioguanine, sulfate, guggulsterone, tolmetin, haloperidol, sulfinpyrazone, chrysin, gleevec, neratinib, and combinations thereof.

22. The therapeutic composition of claim 1, wherein the one or more markers comprises a receptor on a surface of the tumor cells.

23. The therapeutic composition of claim 22, wherein the receptor is selected from the group consisting of epidermal growth factor receptors, cytokine receptors, interleukin receptors, interleukin-13 receptors, interleukin-4 receptors, transferrin receptors, neuropilin receptors, vascular endothelial growth factor receptors, integrins, gastrin-releasing peptide receptors, hepatocyte growth factor receptors, HER-2 receptors, prostate specific membrane antigens, c-met, and combinations thereof.

24. The therapeutic composition of claim 1, wherein the one or more targeting agents are selected from the group consisting of antibodies, proteins, peptides, RNA, DNA, aptamers, small molecules, dendrimers, and combinations thereof.

25. The therapeutic composition of claim 1, wherein the one or more targeting agents comprise an antibody directed against a marker of the tumor cells.

26. The therapeutic composition of claim 1, wherein the one or more targeting agents comprise a peptide directed against a marker of the tumor cells.

27. The therapeutic composition of claim 1, wherein the one or more targeting agents comprise a small molecule directed against a marker of the tumor cells.

28. The therapeutic composition of claim 1, wherein the tumor cells are associated with at least one of cervical cancer, brain cancer, breast cancer, prostate cancer, colorectal cancer, and combinations thereof.

29. The therapeutic composition of claim 1, wherein the tumor cells are associated with brain tumors.

30. The therapeutic composition of claim 1, wherein the tumor cells comprise cancer stem cells.

31. A method of targeting tumor cells in a subject, wherein the method comprises:
administering a therapeutic composition to the subject, wherein the therapeutic composition comprises:
a plurality of nanovectors,
one or more active agents associated with the nanovectors, wherein the one or more active agents have activity against the tumor cells,
one or more active agent enhancers associated with the nanovectors, and
one or more targeting agents associated with the nanovectors, wherein the one or more targeting agents have recognition activity for one or more markers of the tumor cells.

32. The method of claim 31, wherein the one or more active agents and the one or more active agent enhancers are associated with same nanovector molecules.

33. The method of claim 31, wherein the one or more active agents and the one or more active agent enhancers are associated with different nanovector molecules.

34. The method of claim 31, wherein the nanovectors comprise hydrophobic domains and hydrophilic domains,
wherein the one or more active agents and the one or more active agent enhancers are associated with the hydrophobic domains, and
wherein the one or more targeting agents are associated with the hydrophilic domains.

35. The method of claim 31, wherein the nanovectors are selected from the group consisting of single-walled carbon nanotubes, double-walled nanotubes, triple-walled nanotubes, multi-walled nanotubes, ultra-short nanotubes, graphene, graphene nanoribbons, graphite, graphite oxide nanoribbons, carbon black, oxidized carbon black, hydrophilic carbon clusters, graphene quantum dots, and combinations thereof.

36. The method of claim 31, wherein the nanovectors are functionalized with a plurality of solubilizing groups.

37. The method of claim 36, wherein the solubilizing groups are selected from the group consisting of polyethylene glycols, poly(p-phenylene oxide), polyethylene imines, poly(vinyl amines), and combinations thereof.

38. The method of claim 31, wherein the nanovectors comprise an ultra-short single-walled carbon nanotube, wherein the nanotube is functionalized with a plurality of solubilizing groups.

39. The method of claim 31, wherein the nanovectors comprise a polyethylene glycol functionalized hydrophilic carbon clusters (PEG-HCC).

40. The method of claim 31, wherein the one or more active agents are selected from the group consisting of cis-platin, SN-38, vinblastine, daunorubicin, paclitaxel, docetaxel, doxorubicin, epirubicin, vincristine, iadarubicin, mitoxantrone, oxaliplatin, topotecan, etoposide, erlotinib, ethisterone, ethinylestradiol, 1,2,3,4-tetrahydronaphthalene-2,3-diamine, 2,2-dichloro-octahydrocyclohexa 1,3-diaza-2-platinacyclopentane, 2,2-dichloro-hexahydro-naphtho1,3-diaza-2-platinacyclopentane, 4,4-dichloro-3,5-diaza-4-platinatetracycloheptadecahexaene, nitrogen mustards, spermine mustards, estrogen mustards, cholesterol mustards, and combinations thereof.

41. The method of claim 31, wherein the one or more active agent enhancers comprise one or more drug transport pump inhibitors.

42. The method of claim 31, wherein the one or more active agent enhancers are selected from the group consisting of fumitremorgan C, indomethacin, 6-thioguanine, sulfate, guggulsterone, tolmetin, haloperidol, sulfinpyrazone, chrysin, gleevec, neratinib, and combinations thereof.

43. The method of claim 31, wherein the one or more markers comprises a receptor on a surface of the tumor cells.

44. The method of claim 31, wherein the one or more targeting agents are selected from the group consisting of antibodies, proteins, peptides, RNA, DNA, aptamers, small molecules, dendrimers, and combinations thereof.

45. The method of claim 31, wherein the tumor cells are associated with at least one of cervical cancer, brain cancer, breast cancer, prostate cancer, colorectal cancer, and combinations thereof.

46. The method of claim 31, wherein the subject is a human being.

47. The method of claim 31, wherein the administering of the therapeutic composition comprises intravenous administration.

48. A method of formulating a therapeutic composition for targeting tumor cells in a subject, wherein the method comprises:
determining expression levels of one or more markers of the tumor cells; and
formulating the therapeutic composition, wherein the formulated therapeutic composition comprises:
a plurality of nanovectors,
one or more active agents associated with the nanovectors,
wherein the one or more active agents have activity against the tumor cells,
one or more active agent enhancers associated with the nanovectors, and
one or more targeting agents associated with the nanovectors,
wherein the one or more targeting agents have recognition activity for the one or more markers of the tumor cells, and
wherein the one or more targeting agents are selected based on the determined expression levels of the one or more markers of the tumor cells.

49. The method of claim 48, further comprising a step of isolating the tumor cells from the subject;

50. The method of claim 49, wherein the isolating of the tumor cells comprises an excision of a portion of a tumor from the subject.

51. The method of claim 48, further comprising a step of determining susceptibility of the tumor cells to one or more active agents, and selecting the one or more active agents based on the determined susceptibility of the tumor cells to the one or more active agents.

52. The method of claim 51, wherein the susceptibility of the tumor cells to one or more active agents is determined by growing different batches of the tumor cells in the presence of different active agents, and comparing growth rates of the different batches with the growth rate of untreated tumor cells.

53. The method of claim 51, wherein the susceptibility of the tumor cells to one or more active agents is determined in the presence of one or more active agent enhancers.

54. The method of claim 48, wherein the expression levels of the one or more markers of the tumor cells are determined by treating the tumor cells with one or more targeting agents that are specific for the markers.

55. The method of claim 48, wherein the one or more active agents and the one or more active agent enhancers are associated with same nanovector molecules.

56. The method of claim 48, wherein the one or more active agents and the one or more active agent enhancers are associated with different nanovector molecules.

57. The method of claim 48, wherein the nanovectors are selected from the group consisting of single-walled carbon nanotubes, double-walled nanotubes, triple-walled nanotubes, multi-walled nanotubes, ultra-short nanotubes, graphene, graphene nanoribbons, graphite, graphite oxide nanoribbons, carbon black, oxidized carbon black, hydrophilic carbon clusters, graphene quantum dots, and combinations thereof.

58. The method of claim 48, wherein the nanovectors comprise an ultra-short single-walled carbon nanotube, wherein the carbon nanotube is functionalized with a plurality of solubilizing groups.

59. The method of claim 48, wherein the nanovectors comprise a polyethylene glycol functionalized hydrophilic carbon clusters (PEG-HCC).

60. The method of claim 48, wherein the one or more active agents are selected from the group consisting of cis-platin, SN-38, vinblastine, daunorubicin, paclitaxel, docetaxel, doxorubicin, epirubicin, vincristine, iadarubicin, mitoxantrone, oxaliplatin, topotecan, etoposide, erlotinib, ethisterone, ethinylestradiol, 1,2,3,4-tetrahydronaphthalene-2,3-diamine, 2,2-dichloro-octahydrocyclohexa 1,3-diaza-2-platinacyclopentane, 2,2-dichloro-hexahydro-naphtho1,3-diaza-2-platinacyclopentane, 4,4-dichloro-3,5-diaza-4-platinatetracycloheptadecahexaene, nitrogen mustards, spermine mustards, estrogen mustards, cholesterol mustards, and combinations thereof.

61. The method of claim 48, wherein the one or more active agent enhancers comprise one or more drug transport pump inhibitors.

62. The method of claim 48, wherein the one or more active agent enhancers are selected from the group consisting of fumitremorgan C, indomethacin, 6-thioguanine, sulfate, guggulsterone, tolmetin, haloperidol, sulfinpyrazone, chrysin, gleevec, neratinib, and combinations thereof.

63. The method of claim 48, wherein the one or more targeting agents are selected from the group consisting of antibodies, proteins, peptides, RNA, DNA, aptamers, small molecules, dendrimers, and combinations thereof.

64. The method of claim 48, wherein the tumor cells are associated with at least one of cervical cancer, brain cancer, breast cancer, prostate cancer, colorectal cancer, and combinations thereof.

65. The method of claim 48, wherein the tumor cells are associated with brain tumors.

66. The method of claim 48, wherein the subject is a human being.
